(19) United States
(12) Patent Application Publication
(10) Pub. No.: US 2006/0093612 A1
(43) Pub. Date: May 4, 2006

(54) Use of heat shock proteins to enhance efficacy of antibody therapeutics

(76) Inventor: Pramod K. Srivastava, Avon, CT (US)

Correspondence Address: JONES DAY, 222 EAST 41ST ST, NEW YORK, NY, 10017, US

(21) Appl. No.: 10/513,204

(22) PCT Filed: May 2, 2003
(86) PCT No.: PCT/US03/13967

§ 371 Date: Nov. 17, 2005

Related U.S. Application Data
(60) Provisional application No. 60/377,483, filed on May 2, 2002.

(51) Int. Cl. A61K 39/00

(52) U.S. Cl. 424/185.1

(57) ABSTRACT

The present invention relates to methods and pharmaceutical compositions useful for the prevention and treatment of any disease wherein the treatment of such disease would be improved by an enhanced immune response, such as infectious diseases, primary and metastatic neoplastic diseases (i.e., cancer), or neurodegenerative or amyloid diseases. In particular, the contemplated invention is directed to method comprising the administration of heat shock/stress proteins (HSPs) or HSP complexes alone or in combination with each other, in combination with the administration of an immunoreactive reagent. The invention also provides pharmaceutical compositions comprising one or more HSPs or HSP complexes in combination with an immunoreactive reagent. Additionally, the invention contemplates the use of the methods and compositions of the invention to enhance or improve passive immunotherapy and effector cell function.

This application claims the benefit of U.S. Provisional Application No. 60/377,483 filed May 2, 2002, which is incorporated by reference herein in its entirety.

### 1. FIELD OF THE INVENTION

The present invention relates to methods and pharmaceutical compositions useful for the prevention and treatment of any disease wherein the treatment of such disease would be improved by an enhanced immune response, such as infectious diseases, primary and metastatic neoplastic diseases (i.e., cancer), or neurodegenerative or amyloid diseases. In particular, the contemplated invention is directed to methods comprising the administration of heat shock/stress proteins (HSPs) or HSP complexes alone or in combination with each other, in combination with the administration of an immunoreactive reagent. The invention also provides pharmaceutical compositions comprising one or more HSPs or HSP complexes in combination with an immunoreactive reagent. Additionally, the invention contemplates the use of the methods and compositions of the invention to enhance or improve passive immunotherapy and effector cell function.

### 2. BACKGROUND OF THE INVENTION

### 2.1 Immune Responses

An organism's immune system reacts with two types of responses to pathogens or other harmful agents—humoral response and cell-mediated response (See Alberts, B. et al., 1994, Molecular Biology of the Cell. 1195-96). When resting B cells are activated by antigen to proliferate and mature into antibody-secreting cells, they produce and secrete antibodies with a unique antigen-binding site. This antibody-secreting reaction is known as the humoral response. On the other hand, the diverse responses of T cells are collectively called cell-mediated immune reactions. There are two main classes of T cells—cytotoxic T cells and helper T cells. Cytotoxic T cells directly kill cells that are infected with a virus or some other intracellular microorganism. Helper T cells, by contrast, help stimulate the responses of other cells: they help activate macrophages, dendritic cells and B cells, for example (See Alberts, B. et al., 1994, Molecular Biology of the Cell. 1228). Both cytotoxic T cells and helper T cells recognize antigen in the form of peptide fragments that are generated by the degradation of foreign protein antigens inside the target cell, and both, therefore, depend on major histocompatibility complex (MHC) molecules, which bind these peptide fragments, carry them to the cell surface, and present them there to the T cells (See Alberts, B. et al., 1994, Molecular Biology of the Cell. 1228). MHC molecules are typically found in abundance on antigen-presenting cells (APCs).

In addition to the acquired immunity discussed above, innate immunity also plays a role in an organism's immune response. The innate immune system is the first line of defense against disease and provides broad, but relatively nonspecific host defenses that lack the properties of antigenic specificity and immunologic memory that characterize acquired immunity. The effector mechanisms of innate immunity include antimicrobial peptides, granulocytes and phagocytes, natural killer cells, dendritic cells, and the alternative complement pathway, which interact with and control adaptive immune responses. Medzhitov and Janeway, 2000, New England J. Med 343:338-344; Moretta, 2002, Nature Reviews Immunology 2:957-964.

### 2.2 Antigen Presentation

Antigen-presenting cells (APCs), such as macrophages and dendritic cells, are key components of innate and adaptive immune responses. Antigens are generally ‘presented’ to T cells on the surfaces of other cells, the APCs. APCs can trap lymph- and blood-borne antigens and, after internalization and degradation, present antigenic peptide fragments, bound to cell-surface molecules of the major histocompatibility complex (MHC), to T cells. APCs may then activate T cells (cell-mediated response) to clonal expansion, and these daughter cells may either develop into cytotoxic T cells or helper T cells, which in turn activate B (humoral response) cells with the same MHC-bound antigen to clonal expansion and specific antibody production (See Alberts, B. et al., 1994, Molecular Biology of the Cell. 1238-45).

Two types of antigen-processing mechanisms have been recognized. The first type involves uptake of proteins through endocytosis by APCs, antigen fragmentation within vesicles, association with class II MHC molecules and expression on the cell surface. This complex is recognized by helper T cells expressing CD4. The other is employed for proteins, such as viral antigens, that are synthesized within the cell and appears to involve protein fragmentation in the cytoplasm. Peptides produced in this manner become associated with class I MHC molecules and are recognized by cytotoxic T cells expressing CD8 (See Alberts, B. et al., 1994, Molecular Biology of the Cell. 1233-34).

Stimulation of T cells involves a number of accessory molecules expressed by both T cell and APC. Co-stimulatory molecules are those accessory molecules that promote the growth and activation of the T cell. Upon stimulation, co-stimulatory molecules induce release of cytokines, such as interleukin 1 (IL-1) or interleukin 2 (IL-2), interferon, etc., which promote T cell growth and expression of surface receptors (See Paul, 1989, Fundamental Immunology. 109-10).

Normally, APCs are quiescent and require activation for their function. The identity of signals which activate APCs is a crucial and unresolved question (See Banchereau, et al., 1998, Nature 392:245-252; Medzhitov, et al., 1998, Curr. Opin. Immunol. 10:12-15).

### 2.3 Passive Immunotherapy

Passive immunotherapy (also termed passive immunization) refers to the administration of an immunoreactive reagent, e.g., a molecule comprising an antigen binding region directed against an epitope on a pathogen, tumor or pathogenic protein and a domain with an Fc receptor-binding region, complement binding region or region that mediates effector cell effects, such as an antibody, directly to a patient. The immunoreactive reagent can be given prophylactically to, for example, inhibit infection, or therapeutically to reduce or eliminate infection, to reduce or eliminate cancer cells, or to clear or remove pathogenic proteins, e.g., protein aggregates or deposits, as occurs in neurodegenerative and/or amyloidogenic disease. Passive immunotherapy is distinguished from active immunotherapy which involves immunization of a patient with an antigen to induce an in vivo immune response, e.g., to produce antibodies or cytotoxic T lymphocytes. Rather, in passive immunotherapy, an immunoreactive reagent, e.g., an antibody is administered to patients and results in the stimulation of effector cells, e.g., cells with Fc receptors capable of interacting with the Fc portion (i.e., the Fc receptor binding region) of the administered antibody or other immunoreactive reagent, resulting in cellular immune functions such as antibody-dependent cellular cytotoxicity (e.g., ADCC) or antibody-mediated opsonization and/or phagocytosis directed against the cell, pathogen, or protein possessing the epitope recognized by the antibody. The efficacy of antibody-mediated tumor therapy which depends on FcR effector cell functions can be modified by the use of specific cytokines. Keler, et al., 2000, J. Immunol. 164:5746-5752. Therapeutic and/or prophylactic antibodies include but are not limited to antibodies that bind to cell surface molecules and antagonize normal function (e.g., blocking antibodies), antibodies that bind to cell surface molecules and mimic normal function (antibody agonists), and sequestering antibodies.

### 2.4 CTLA-4

Cytotoxic T Lymphocyte Antigen-4 (CTLA-4) is a glycoprotein which is expressed on the surface of activated T cells at low levels. CTLA-4 is similar to CD28, and has a greater binding affinity for B7 family members (e.g. B7-1 and B7-2) than CD28. Binding of CTLA-4 on T lymphocytes to the B7 ligand mediates a negative signal, inhibiting IL-2 secretion and cellular proliferation. In sum, CTLA-4-mediated inhibition of T cell activation results in a “switching off” of T-cell regulated immune responses, particularly early T-cell activation events. (Brunner, et al., 1999 J. Immunol. 162:5813-5820.)

It was recently discovered that the blockade of CTLA-4 function via treatment with anti-CTLA-4 antibodies, resulted in the enhancement of various immune responses and contributed toward the inducement of tumor immunity (Leach, et al., 1996, Science 271:1734-1736; PCT publications WO 00/322231 and WO 01/14424). Mice transplanted with B16 melanoma cells showed tumor regression and elevated levels of CD8⁺ T cells upon combination treatment with an anti-CTLA-4 mAb and a GM-CSF-producing tumor cell vaccine. It was noted that administration of this combination treatment in a prophylactic setting (i.e., prior to tumor challenge), resulted in full protection even in the absence of CD8⁺ T cells. Data demonstrated that therapeutic autoreactive CD8⁺ T cells can be induced in tumor-bearing mice. (Van Elsas, et al., 2001, J. Exp. Med. 194:481-489). Co-administration of an anti-CTLA-4 antibody in combination with a GM-CSF tumor cell vaccine demonstrated efficacy against established B16-BL6 melanoma cells, but little effect was noted when either therapy was administered alone. (Van Elsas, et al., 1999, J. Exp. Med. 190:355-366). In addition, it has also been noted that blockade of CTLA-4 correlates with an enhancement of helper function and induced amplification of CD4⁺ T cells. (Hernandez, et al., 2001, J. Immun. 3908-3914). It has been observed that blockade via the administration of an anti-CTLA-4 antibody resulted in enhanced host resistance to a intracellular pathogen, an increase in the number of IFN-g and IL-4 producing cells in the liver and spleen, and an enhanced resulting hepatic granulomatous response (Murphy, et al., 1998, J. Immun. 4153-4160).

### 2.5 Heat-Shock Proteins

Heat shock proteins (HSPs), which are also referred to interchangeably herein as stress proteins, can be selected from among any cellular protein that satisfies the following criteria. It is a protein whose intracellular concentration increases when a cell is exposed to a stressful stimuli, it is capable of binding other proteins or peptides, and it is capable of releasing the bound proteins or peptides in the presence of adenosine triphosphate (ATP) or low pH. In addition, HSPs include constitutively expressed conserved cellular homologs of the proteins induced by stress. It has been discovered that the Hsp-60, Hsp-70 and Hsp-90 families are composed of proteins related to the stress proteins in amino acid sequence, for example, having greater than 35% amino acid identity, but whose expression levels are not altered by stressful stimuli. Accordingly, it is contemplated that the definition of stress protein, as used herein, embraces other proteins, muteins, analogs, and variants thereof having at least 35% to 55%, preferably 55% to 75%, and most preferably 75% to 85% amino acid identity with members of the three families whose expression levels in a cell are stimulated in response to stressful stimuli.

The first stress proteins to be identified were the HSPs. As their name implies, HSPs are synthesized by a cell in response to heat shock. To date, three major families of HSPs have been identified based on molecular weight. The families have been called hsp60, hsp70 and hsp90 where the numbers reflect the approximate molecular weight of the stress proteins in kilodaltons. Many members of these families were found subsequently to be induced in response to other stressful stimuli including, but not limited to, nutrient deprivation, metabolic disruption, oxygen radicals, and infection with intracellular pathogens. (See Welch, May 1993, Scientific American 56-64; Young, 1990, Annu. Rev. Immunol. 8:401-420; Craig, 1993, Science 260:1902-1903; Gething, et al., 1992, Nature 355:33-45; and Lindquist, et al., 1988, Annu. Rev. Genetics 22:631-677), the disclosures of which are incorporated herein by reference. It is contemplated that hsps/stress proteins belonging to all of these three families can be used in the practice of the instant invention.

HSPs are intracellular molecules that are abundant, soluble, and highly conserved. As intracellular chaperones, HSPs participate in many biochemical pathways of protein maturation and function active during times of stress and normal cellular homeostasis. Many stresses can disrupt the three-dimensional structure, or folding, of a cell's proteins. Left uncorrected, mis-folded proteins form aggregates that may eventually kill the cell. HSPs bind to those damaged proteins, helping them refold into their proper conformations. In normal (unstressed) cellular homeostasis, HSPs are required for cellular metabolism. HSPs help newly synthesized polypeptides fold and thus prevent premature interactions with other proteins. Also, HSPs aid in the transport of proteins throughout the cell's various compartments.

The major HSPs can accumulate to very high levels in stressed cells, but they occur at low to moderate levels in cells that have not been stressed. For example, the highly inducible mammalian hsp70 is hardly detectable at normal temperatures but becomes one of the most actively synthesized proteins in the cell upon heat shock (Welch, et al., 1985, J. Cell. Biol. 101:1198-1211). In contrast, hsp90 and hsp60 proteins are abundant at normal temperatures in most, but not all, mammalian cells and are further induced by heat (Lai, et al., 1984, Mol. Cell. Biol. 4:2802-2810; van Bergen en Henegouwen, et al., 1987, Genes Dev. 1:525-531).

HSPs have been found to have immunological and antigenic properties. Immunization of mice with gp96 or p84/86 isolated from a particular tumor rendered the mice immune to that particular tumor, but not to antigenically distinct tumors (Srivastava, P. K., et al., 1988, Immunogenetics 28:205-207; Srivastava, P. K., et al., 1991, Curr. Top. Microbiol. Immunol. 167:109-123). Further, hsp70 was shown to elicit immunity to the tumor from which it was isolated but not to antigenically distinct tumors. However, hsp70 depleted of peptides was found to lose its specific immunogenic activity (Udono, M., and Srivastava, P. K., 1993, J. Exp. Med. 178:1391-1396). These observations suggested that the heat shock proteins are not antigenic per se, but form noncovalent complexes with antigenic peptides, and the complexes can elicit specific immunity to the antigenic peptides (Srivastava, P. K., 1993, Adv. Cancer Res. 62:153-177; Udono, H., et al., 1994, J. Immunol. 152:5398-5403; Suto, R., et al., 1995, Science 269:1585-1588). Recently, hsp60 and hsp70 have been found to stimulate production of proinflammatory cytolines, such as TNFα and IL-6, by monocytes, macrophages, or cytotoxic T cells (Breloer et al., 1999, J. Immunol. 162:3141-3147; Chen et al., 1999, J. Immunol. 162:3212-3219; Ohashi et al., 2000, J. Immunol. 164:558-561; Asea et al., 2000, Nature Medicine, 6:435-442; Todryk et al., 1999, J. Immunol. 163:1398-1408). Hsp70 has also been shown to target immature dendritic cells and make them more able to capture antigens (Todryk et al., 1999, J. Immunol. 163:1398-1408). It has been postulated that release of or induction of expression of hsp60 and hsp70, e.g., due to cell death, may serve to signal that an immune reaction should be raised (Chen et al., 1999, J. Immunol. 162:3212-3219; Ohashi et al., 2000, J. Immunol. 164:558-561; Todryk et al., 1999, J. Immunol. 163:1398-1408; Basu et al., 2000, Intl. Immunol. 12: 1539-1546).

The use of noncovalent complexes of HSP and peptide, purified from cancer cells, for the treatment and prevention of cancer has been described in U.S. Pat. Nos. 5,750,119, 5,837,251, and 6,017,540.

The use of HSP-peptide complexes for sensitizing antigen presenting cells in vitro-for use in adoptive immunotherapy is described in U.S. Pat. Nos. 5,985,270 and 5,830,464.

HSP-peptide complexes can also be isolated from pathogen-infected cells and used for the treatment and prevention of infection caused by the pathogen, such as viruses, and other intracellular pathogens, including bacteria, protozoa, fungi and parasites; see U.S. Pat. Nos. 5,961,979, and 6,048,530.

Immunogenic HSP-peptide complexes can also be prepared by in vitro complexing of HSPs and antigenic peptides, and the uses of such complexes for the treatment and prevention of cancer and infectious diseases has been described in U.S. Pat. Nos. 5,935,576, and 6,030,618. The use of heat shock protein in combination with a defined antigen for the treatment of cancer and infectious diseases have also been described in PCT publication WO97/06821 dated Feb. 27, 1997.

The purification of HSP-peptide complexes from cell lysate has been described previously; see for example, U.S. Pat. Nos. 5,750,119, and 5,997,873.

### 3. SUMMARY OF THE INVENTION

The present invention relates to methods and compositions useful for producing or enhancing an immune response comprising administering a heat shock protein (HSP) preparation with an immunoreactive reagent. The methods and compositions are useful for improving the treatment outcome in a subject administered the HSP preparation alone and/or the immunoreactive reagent alone. In particular, the invention provides methods and compositions useful for producing or enhancing an immune response elicited by an immunoreactive reagent, and/or improving the efficacy of an immunoreactive reagent, comprising the administration of an HSP preparation. Accordingly, the methods and compositions encompass administering an HSP preparation to enhance passive immunotherapy. In specific embodiments, such methods and compositions comprise administering an HSP preparation and are useful for enhancing the immunoreactive reagent's ability to stimulate effector cell function. The present invention also contemplates methods and compositions useful for enhancing an immune response elicted by an HSP preparation, comprising the administration of an immunoreactive reagent. Given the invention, the methods and compositions of the invention are useful for the prevention and treatment of diseases and disorders wherein the treatment or prevention would be improved by an enhanced immune response, such as infectious diseases, primary and metastatic neoplastic diseases (i.e., cancer), neurodegenerative or amyloid diseases, or protein deposition or amyloidogenic diseases. Thus, the invention encompasses methods and compositions designed to treat or prevent infectious diseases, primary and metastatic neoplastic diseases (i.e., cancer), neurodegenerative or amyloid diseases, or protein deposition or amyloidogenic diseases comprising administering one or more immunoreactive reagents in combination with an HSP preparation.

In one embodiment, the invention provides a method of producing or increasing an immune response elicited by an immunoreactive reagent by using an HSP preparation, wherein the HSP preparation enhances an immune response by an amount of immunoreactive reagent which is otherwise sub-optimal for inducing the immune response when used alone. In certain embodiments, when the HSP preparation is not used in conjunction with an immunoreactive reagent to elicit an immune response, administering said HSP preparation alone does not produce or increase said immune response. In alternate embodiments, both the HSP preparation and the immunoreactive reagent can elicit an immune response alone and/or when administered in combination.

In certain embodiments, the HSP preparation may enhance the effects of the immunoreactive reagent in an additive manner. In a preferred embodiment, the HSP preparation enhances the effects of the immunoreactive reagent in a synergistic manner. In another embodiment, the immunoreactive reagent enhances the effect of an HSP preparation in an additive manner. Preferably, the effects are enhanced in a synergistic manner. Thus, in certain embodiments, the invention encompasses methods of disease treatment or prevention that provide better therapeutic profiles than administration of HSP preparation alone and/or immunoreactive reagent alone. Encompassed by the invention are combination therapies that have additive potency or an additive therapeutic effect while reducing or avoiding unwanted or adverse effects. The invention also encompasses synergistic combinations where the therapeutic efficacy is greater than additive, while unwanted or adverse effects are reduced or avoided. In certain embodiments, the methods of the invention permit treatment or prevention of diseases and disorders wherein treatment is improved by an enhanced immune response using lower and/or less frequent doses of immunoreactive reagents and/or HSPs to reduce the incidence of unwanted or adverse effects caused by the administration of immunoreactive agents and/or HSPs alone, while maintaining or enhancing efficacy of treatment, preferably increasing patient compliance, improving therapy and/or reducing unwanted or adverse effects.

The methods and compositions of the invention are useful not only in untreated patients but are also useful in the treatment of patients partially or completely un-responsive to HSPs administered alone or immunoreactive reagents administered alone. In various embodiments, the invention provides methods and compositions useful for the treatment of diseases or disorders in patients that have been shown to be or may be refractory or non-responsive to therapies comprising the administration of either or both immunoreactive reagents and/or HSPs, and wherein treatment is improved by an enhanced immune response.

HSP preparations useful in the methods and compositions of the invention can include, but are not limited to, free HSP(s) not bound to any molecule, and molecular complexes of HSP with another molecule, such as a peptide. An HSP-peptide complex comprises an HSP covalently or noncovalently attached to a peptide. The HSP-peptide complex may consist of HSPs bound to peptides derived from the tumor, pathogen or cell type and /or protein of interest. In one embodiment, said peptide is the same target recognized by immunoreactive reagent or is derived from the tumor, pathogen or cell type and /or protein of interest. Alternately, the HSP-peptide complex may consist of HSPs bound to an endogenous peptide, but not necessarily a peptide from the same source as the target of the immunoreactive reagent. Certain methods of the invention would not require covalent or noncovalent attachment of HSPs to any specific antigens or antigenic peptides prior to administration to a subject. The HSP preparations useful in the methods and compositions of the invention also include HSP fusion proteins. The HSP fusion proteins may consist of HSPs fused to any antigenic peptide sequence wherein the peptide sequence is derived from the tumor, pathogen or cell type and/or protein of interest. In one embodiment, said peptide sequence is the same target recognized by the immunoreactive reagent or is derived from the tumor, pathogen or cell type and/or protein of interest.

Immunoreactive reagents useful in the methods and compositions of the invention can include, but are not limited to, antibodies, molecules or proteins engineered to include the antigen binding portion of an antibody, molecules or proteins engineered to include an antigen binding domain that mediates antibody dependent immune responses, a peptide or domain that interacts specifically with the antigen of interest, or a molecule that has any antigen binding domain that interacts with an antigen/epitope of interest and the domain of the constant region of an antibody that mediates antibody dependent immune responses, such as effector cell responses or processes. The antigen binding domain recognizes a specific target and the domain of a constant region mediates antibody dependent immune effector cell responses.

In preferred embodiments, the immunoreactive agent is an antibody, preferably with in vivo therapeutic or prophylactic uses and the invention provides methods and compositions useful for enhancing the efficacy of such therapeutic or prophylactic antibodies comprising the administration of an HSP preparation. In certain embodiments, the antibody's ability to stimulate effector cell function is enhanced by administering an HSP preparation. In a specific embodiment, antibody dependent cellular cytotoxicity and/or phagocytosis of tumor cells or pathogens or pathogenic proteins and peptides is enhanced by use of a therapeutic antibody in combination with an HSP preparation. Preferably the therapeutic antibody is a cytotoxic and/or opsonizing antibody. Accordingly, the invention provides methods and compositions wherein an HSP preparation is used in combination with an immunoreactive reagent to enhance effector cell function (i.e., antibody dependent cellular cytotoxicity and phagocytosis) for macrophages, natural killer (NK) cells and polymorphonuclear cells. Preferably the immunoreactive reagent is an antibody, more preferably a cytotoxic and/or opsonizing antibody. In one embodiment, the HSP-mediated enhancement of passive immunotherapy occurs through stimulation of effector cells, i.e., induction and/or activation of the Fc receptors on such cells.

Antibodies used in the methods of the invention include, but are not limited to, monoclonal antibodies, polyclonal antibodies, synthetic antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies used in the methods of the present invention include immunoglobulin molecules and immunologically active portions of immunoglobulin molecules, i.e., molecules that contain an antigen binding site that immunospecifically binds to the target of interest. The immunoglobulin molecules of the invention can be of any type (e.g. IgG, IgE, IgM, IgD, IgA and IgY), class (e.g., IgG₁, IgG₂, IgG₃, IgG₄, IgA₁ and IgA₂) or subclass of immunoglobulin molecule.

Without being bound by any theory, an increased concentration of HSP may induce production of cytokines and surface expression of antigen-presenting and co-stimulatory molecules. Accordingly, the HSP preparation administered to a subject can boost the effectiveness of an immunoreactive reagent by increasing the efficiency and effectiveness of antigen presentation.

In other preferred embodiments, an immunoreactive reagent is administered to a subject receiving an HSP preparation to improve the treatment outcome. In a specific embodiment, the immunoreactive reagent enhances the immune response elicited by the administration of the HSP preparation.

In a specific embodiment of the invention, the antibody is an anti-CTLA-4 antibody. In another specific embodiment, the antibody is an anti-c-erb-2 antibody, preferably human rhu 4D5 (Herceptin) particularly useful in treating or preventing cancers that express the Her2/neu oncogene. In yet another specific embodiment, the antibody is anti-tumor MoAb (MS11G6), an IgG2a anti-idiotype antibody, useful in therapy for cancers such as but not limited to NK-cell-resistant lymphoma

In one embodiment, an HSP preparation is administered in combination with anti-tumor or anti-cancer antibody therapy directed against a cancer. In an alternate embodiment, an HSP preparation is administered in combination with antibody therapy directed against a pathogen. In yet another embodiment, an HSP preparation is administered in combination with antibody therapy directed against a pathogenic or unwanted protein or a cell affected by neurodegenerative or amyloid disease or disorder.

In other embodiments, the methods and compositions of the invention can be used to generate an immune response against epitopes associated with neurodegenerative or amyloid diseases, cancer or an agent of infectious disease or any component, cell or molecule bearing an epitope associated with the aforementioned diseases, by administering to an individual a therapeutic amount of the immunoreactive reagent and an HSP preparation. Where an immune response against a type of cancer is desired, an immunoreactive reagent is used that specifically binds to (or “recognizes”) an antigen of the type of cancer, e.g., a tumor-associated antigen. In other embodiments, the methods and compositions of the invention comprise administration of an immunoreactive reagent that specifically binds to an antigen of a type of cancer in combination with an HSP preparation for the treatment or prevention of said type of cancer. Where eliciting an immune response against an agent of an infectious disease is desired, an immunoreactive reagent which specifically binds to an antigen or pathologic protein (e.g., toxin) of the agent of infectious disease is administered. In alternate embodiments, the methods and compositions of the invention comprise the administration of an immunoreactive reagent that specifically binds to an agent of an infectious disease in combination with an HSP preparation to treat or prevent said infectious disease. In yet other embodiments, the methods and compositions of the invention comprise administration of an immunoreactive reagent that specifically binds an antigenic molecule associated with a neurodegenerative disease or an amyloid disease in combination with an HSP preparation to treat or prevent said neurodegenerative or amyloid disease. Preferably, the immunoreactive reagent is an antibody.

The invention also includes methods and compositions comprising administration of an HSP preparation in combination with an immunoreactive reagent to patients that have previously received or are currently receiving other forms of medical therapy, including anti-cancer agents, antibiotics, and anti-infectious agents.

In another embodiment, the invention provides a method of activating antigen presenting cells comprising contacting APCs with an HSP preparation and administering such activated APCs in combination with the administration of an immunoreactive reagent. Accordingly, the invention provides methods and compositions for enhancing the immune response elicited by an immunoreactive reagent comprising administration of activated APCs and/or an HSP preparation. Preferably, the HSP preparation does not efficiently elicit an immune response in the absence of the administration of the immunoreactive reagent. In certain embodiments, the HSP preparation does not display the immunogenicity of the target recognized by the immunoreactive reagent. In alternate embodiments, the immunogenicity of the HSP preparation displays the immunogenicity of the target recognized by the immunoreactive reagent. The immunogenicity of an HSP preparation can be tested in vivo or in vitro by any method known in the art.

In specific embodiments, the methods and compositions of the invention comprising administration of an immunoreactive reagent with administration of activated APCs and/or an HSP preparation are useful for the treatment of any disease or disorder wherein the treatment of such disease would be improved by an enhanced immune response, in particular, an antibody mediated immune response, such as but not limited to infectious diseases, cancer, or neurodegenerative or amyloid diseases or disorders.

Also encompassed by the invention are methods of delivering one or more HSPs as adjunctive therapy in combination with immunoreactive reagents; pharmaceutical compositions and formulas for administration comprising one or more HSP preparations and one or more immunoreactive reagents, kits comprising said pharmaceutical compositions; and methods of treating or preventing a disease that would be improved by an enhanced immune response, such as infectious diseases, primary and metastatic neoplastic diseases (i.e., cancer), neurodegenerative or amyloid diseases, or protein deposition or amyloidogenic diseases, using the prophylactic or therapeutic pharmaceutical compositions of the invention. Such methods, kits and compositions can further include the administration of activated APCs.

### 4. DETAILED DESCRIPTION OF THE INVENTION

The present invention relates to methods and compositions useful for producing or enhancing an immune response comprising administering a heat shock protein (HSP) preparation with an immunoreactive reagent. The methods and compositions are useful for improving the treatment outcome in a subject administered the HSP preparation alone and/or the immunoreactive reagent alone. In particular, the invention provides methods and compositions for improving the prophylactic or therapeutic efficacy of an immunoreactive agent. The invention also provides methods and compositions useful for producing or enhancing an immune response elicited by an immunoreactive reagent, comprising the administration of an HSP preparation. Accordingly, the methods and compositions encompass administering an HSP preparation to enhance passive immunotherapy. In specific embodiments, such methods and compositions comprise administering an HSP preparation and are useful for enhancing the immunoreactive reagent's ability to stimulate effector cell function. The present invention also contemplates methods and compositions useful for enhancing an immune response elicted by an HSP preparation, comprising the administration of an immunoreactive reagent. Given the invention, the methods and compositions of the invention are useful for the prevention and treatment of diseases and disorders wherein the treatment or prevention would be improved by an enhanced immune response, such as infectious diseases, primary and metastatic neoplastic diseases (i.e., cancer), neurodegenerative or amyloid diseases, or protein deposition or amyloidogenic diseases. Thus, the invention encompasses methods and compositions designed to treat or prevent infectious diseases, primary and metastatic neoplastic diseases (i.e., cancer), neurodegenerative or amyloid diseases, or protein deposition or amyloidogenic diseases comprising administering one or more immunoreactive reagents in combination with an HSP preparation.

In one embodiment, the invention provides a method of producing or increasing an immune response elicited by an immunoreactive reagent by using an HSP preparation, wherein the HSP preparation facilitates the induction of an immune response by an amount of immunoreactive reagent which is otherwise insufficient for inducing the immune response when used alone. In certain embodiments, when the HSP preparation is not used in conjunction with an immunoreactive reagent to elicit an immune response, administering said HSP preparation alone does not produce or increase said immune response. In alternate embodiments, both the HSP preparation and the immunoreactive reagent can elicit an immune response alone and/or when administered in combination.

In certain embodiments, the HSP preparation may enhance the effects of the immunoreactive reagent in an additive manner. In a preferred embodiment, the HSP preparation enhances the effects of the immunoreactive reagent in a synergistic manner. In another embodiment, the immunoreactive reagent enhances the effect of an HSP preparation in an additive manner. Preferably, the effects are enhanced in a synergistic manner. Thus, in certain embodiments, the invention encompasses methods of disease treatment or prevention that provide better therapeutic profiles than administration of HSP preparation alone and/or immunoreactive reagent alone. Encompassed by the invention are combination therapies that have additive potency or an additive therapeutic effect while reducing or avoiding unwanted or adverse effects. The invention also encompasses synergistic combinations where the therapeutic efficacy is greater than additive, while unwanted or adverse effects are reduced or avoided. In certain embodiments, the methods of the invention permit treatment or prevention of diseases and disorders wherein treatment is improved by an enhanced immune response using lower and/or less frequent doses of immunoreactive reagents and/or HSPs to reduce the incidence of unwanted or adverse effects caused by the administration of immunoreactive agents and/or HSPs alone, while maintaining or enhancing efficacy of treatment, preferably increasing patient compliance, improving therapy and/or reducing unwanted or adverse effects.

The methods and compositions of the invention are useful not only in untreated patients but are also useful in the treatment of patients partially or completely un-responsive to HSPs administered alone or immunoreactive reagents administered alone. In various embodiments, the invention provides methods and compositions useful for the treatment of diseases or disorders in patients that have been shown to be or may be refractory or non-responsive to therapies comprising the administration of either agent alone, and wherein treatment is improved by an enhanced immune response.

HSP preparations useful in the methods and compositions of the invention can include, but are not limited to, free HSP(s) not bound to any molecule, and molecular complexes of HSP with another molecule, such as a peptide. An HSP-peptide complex comprises an HSP covalently or noncovalently attached to a peptide. The HSP-peptide complex may consist of HSPs bound to peptides derived from the tumor, pathogen or cell type and/or protein of interest, preferably said peptide is the same target recognized by the immunoreactive reagent. Alternately, the HSP-peptide complex may consist of HSPs bound to an endogenous peptide, but not necessarily a peptide from the same source as the target of the immunoreactive reagent. Certain methods of the invention would not require covalent or noncovalent attachment of HSPs to any specific antigens or antigenic peptides prior to administration to a subject. The invention encompasses use of HSP-peptide complexes comprising HSPs covalently or non-covalently complexed to exogenous peptides, produced in vitro as well as use of endogenous HSP-peptide complexes isolated from cellular sources.

Immunoreactive reagents useful in the methods and compositions of the invention can include, but are not limited to, antibodies, molecules or proteins engineered to include the antigen binding portion of an antibody, molecules or proteins engineered to include an antigen binding domain that mediates antibody dependent immune responses, a peptide or domain that interacts specifically with the antigen of interest, or any antigen binding domain that interacts with an antigen/epitope of interest. The antigen binding domain is preferably associated with a domain of the constant region of an antibody that mediates antibody dependent immune response, immune effector cell responses or processes. Preferably the immunoreactive reagent is purified.

In preferred embodiments, the immunoreactive agent is an antibody, preferably with in vivo therapeutic or prophylactic uses and the invention provides methods and compositions useful for enhancing the efficacy of such therapeutic or prophylactic antibodies comprising the administration of an HSP preparation. In such embodiments, the antibody's ability to stimulate effector cell function is enhanced by administering an HSP preparation. In a specific embodiment, antibody dependent cellular cytotoxicity and/or phagocytosis of tumor cells or pathogens or pathogenic proteins and peptides is enhanced by use of a therapeutic antibody in combination with an HSP preparation. Preferably the therapeutic antibody is a cytotoxic and/or opsonizing antibody. Accordingly, the invention provides methods and compositions wherein an HSP preparation is used in combination with an immunoreactive reagent to enhance effector cell function (i.e., antibody dependent cellular cytotoxicity and phagocytosis) for macrophages, natural killer (NK) cells and polymorphonuclear cells. Preferably the immunoreactive reagent is an antibody, more preferably a cytotoxic and/or opsonizing antibody. In one embodiment, the HSP-mediated enhancement of passive immunotherapy occurs through stimulation of effector cells, i.e., induction and/or activation of the Fc receptors on such cells.

In other preferred embodiments, an immunoreactive reagent is administered to a subject receiving an HSP preparation to improve the treatment outcome. In a specific embodiment, the immunoreactive reagent enhances the immune response elicited by the administration of the HSP preparation. In other embodiments, the immunoreactive reagent potentiates T cell activation elicited by HSPs.

In a specific embodiment of the invention, the antibody is an anti-CTLA-4 antibody. In another specific embodiment, the antibody is an anti-c-erb-2 antibody, preferably human rhu 4D5 (Herceptin) particularly useful in treating or preventing cancers that express the Her2/neu oncogene. In another specific embodiment, the antibody is anti-tumor MoAb (MS11G6), an IgG2a anti-idiotype antibody, useful in therapy for cancers such as but not limited to NK-ell-resistant lymphoma. In one embodiment, the antibody is an agonist of a Toll-Like Receptor (TLR), e.g., TLR 2, 7, 8, or 9. In another embodiment, the antibody is an agonist of 41BB (see e.g., Miller et al., 2002, J. Immunol. 169:1792-1800), OX40, ICOS, or CD40. In yet another embodiment, the antibody is an antagonist of Fas ligand or PD1. In another embodiment, the antibody is Mab 6B11 which binds to the CDR3 loop of the T cell receptor (TCR) of invariant NKT cells and expands and/or activates these cells. See US 2002/0164331 published Nov. 7, 2002.

In one embodiment, an HSP preparation is administered in combination with anti-tumor antibody therapy directed against a cancer. In an alternate embodiment, an HSP preparation is administered in combination with antibody therapy directed against a pathogen. In yet another embodiment, an HSP preparation is administered in combination with antibody therapy directed against a cell affected by neurodegenerative or amyloid disease or disorder.

Without being bound by any theory, an increased concentration of HSP may induce production of cytokines and surface expression of antigen-presenting and co-stimulatory molecules. Accordingly, it is believed that the HSP preparation administered to a subject can boost the effectiveness of an immunoreactive reagent by increasing the efficiency and effectiveness of antigen presentation. In certain embodiments, it is believed that the HSP preparation can enhance antibody-mediated responses such as cell effector functions.

In other embodiments, the methods and compositions of the invention can be used to generate an immune response against epitopes associated with neurodegenerative or amyloid diseases, cancer or an agent of infectious disease or any component, cell or molecule bearing an epitope associated with the aforementioned diseases, by administering to an individual a therapeutic amount of the immunoreactive reagent and an HSP preparation. Where an immune response against a type of cancer is desired, an immunoreactive reagent is used that specifically binds to (or “recognizes”) an antigen of the type of cancer, i.e., displays the immunogenicity of a cancer. Examples of such antigens are a tumor-associated antigen (i.e., relatively overexpressed in tumor cells) or a tumor specific antigen (i.e., only present in tumor cells). In other embodiments, the methods and compositions of the invention comprise administration of an immunoreactive reagent that specifically binds to an antigen of a type of cancer in combination with an HSP preparation for the treatment or prevention of said type of cancer. Where eliciting an immune response against an agent of an infectious disease is desired, an immunoreactive reagent which specifically binds to an antigen or pathologic protein (e.g., toxin) of the agent of infectious disease is administered. In alternate embodiments, the methods and compositions of the invention comprise the administration of an immunoreactive reagent that specifically binds to an agent of an infectious disease in combination with an HSP preparation to treat or prevent said infectious disease. In yet other embodiments, the methods and compositions of the invention comprise administration of an immunoreactive reagent that specifically binds an antigenic molecule or protein epitope associated with a neurodegenerative disease or an amyloid disease in combination with an HSP preparation to treat or prevent said neurodegenerative or amyloid disease. Preferably, the immunoreactive reagent is an antibody.

The invention also includes methods and compositions comprising administration of an HSP preparation in combination with an immunoreactive reagent to patients that have previously received or are currently receiving other forms of medical therapy, including anti-cancer agents, antibiotics, and anti-infectious agents.

In another embodiment, the invention provides a method of activating antigen presenting cells comprising contacting APCs with an HSP preparation and administering such activated APCs in combination with the administration of an immunoreactive reagent. Accordingly, the invention provides methods and compositions for enhancing the immune response elicited by an immunoreactive reagent comprising administration of activated APCs and/or an HSP preparation. Preferably, the HSP preparation does not efficiently elicit an immune response in the absence of the administration of the immunoreactive reagent. In certain embodiments, the HSP preparation does not display the immunogenicity of the target recognized by the immunoreactive reagent. In alternate embodiments, the immunogenicity of the HSP preparation displays the immunogenicity of the target recognized by the immunoreactive reagent. The immunogenicity of an HSP preparation can be tested in vivo or in vitro by any method known in the art.

In specific embodiments, the methods and compositions of the invention comprising administration of an immunoreactive reagent with administration of activated APCs and/or an HSP preparation are useful for the treatment of any disease or disorder wherein the treatment of such disease would be improved by an enhanced immune response, such as but not limited to infectious diseases, cancer, or neurodegenerative or amyloid diseases or disorders.

Also encompassed by the invention are methods of delivering one or more HSPs as adjunctive therapy in combination with immunoreactive reagents; pharmaceutical compositions and formulas for administration comprising one or more HSP preparations and one or more immunoreactive reagents, kits comprising said pharmaceutical compositions; and methods of treating or preventing a disease that would be improved by an enhanced immune response, such as infectious diseases, primary and metastatic neoplastic diseases (i.e., cancer), neurodegenerative or amyloid diseases, or protein deposition or amyloidogenic diseases, using the prophylactic or therapeutic pharmaceutical compositions of the invention. Such methods, kits and compositions can further include the administration of activated APCs.

### 4.1 Prophylactic/Therapeutic Methods

The present invention provides methods for producing or increasing an immune response elicited by an immunoreactive reagent, comprising the administration of an HSP preparation in conjunction with the administration of an immunoreactive reagent. The present invention encompasses methods for treating or preventing diseases and disorders wherein the treatment or prevention would be improved by an enhanced immune response. In preferred embodiments, an enhanced immune response includes enhancement of responses such as such as antibody-dependent cellular cytotoxicity (e.g., ADCC) or antibody-mediated opsonization and/or phagocytosis directed against the cell, pathogen, or protein possessing the epitope recognized by the antibody and complement mediated cell killing by acting on effector cell mechanisms. In certain embodiments, the HSP preparation induces T-cell activation and the immunoreactive reagent, e.g., an antibody, can enhance the immune response by potentiating T cell activation.

In one embodiment, “treatment” or “treating” refers to an amelioration of cancer, an infectious disease, or a neurodegenerative or amyloid disease, or at least one discernible symptom thereof. In another embodiment, “treatment” or “treating” refers to an amelioration of at least one measurable physical parameter associated with cancer, an infectious disease, a neurodegenerative or amyloid disease, not necessarily discernible by the subject. In yet another embodiment, “treatment” or treating” refers to inhibiting the progression of a cancer, an infectious disease, a neurodegenerative or amyloid disease, either physically, e.g., stabilization of a discernible symptom, physiologically, e.g., stabilization of a physical parameter, or both. In yet another embodiment, “treatment” or “treating” refers to delaying the onset of a cancer, a neurodegenerative or amyloid disease.

In certain embodiments, the methods and compositions of the present invention are useful as a preventative measure against cancer, an infectious disease, a neurodegenerative or amyloid disease. As used herein, “prevention” or “preventing” refers to a reduction of the risk of acquiring a given cancer, infectious disease, neurodegenerative or amyloid disease. In one mode of the embodiment, the methods and compositions of the present invention encompass administration of an HSP preparation with administration of an immunoreactive reagent as a preventative measure to a human subject having a genetic predisposition to a cancer, infectious disease, neurodegenerative or amyloid disease. In another mode of the embodiment, the methods and compositions of the invention are useful as a preventative measure to a subject having a non-genetic predisposition to a cancer, or to a subject facing exposure to an agent of an infectious disease.

In other embodiments, the methods and compositions of the present invention are useful for treating or preventing the clinical manifestation or onset of cancer, an infectious disease or neurodegenerative or amyloid disease.

In certain embodiments, the invention provides methods for treating or preventing infectious diseases, cancer, neurodegenerative or amyloid diseases, or protein deposit or amyloid diseases comprising administration of an HSP preparation in combination with one or more immunoreactive reagents. In certain embodiments, an HSP preparation is administered to a mammal, preferably a human, concurrently with one or more immunoreactive reagents.

In one embodiment, the HSP preparation and immunoreactive reagent are administered simultaneously. In another embodiment, the HSP preparation and the immunoreactive reagent are administered to a subject in a sequence and within a time interval such that the HSP preparation can act together with the immunoreactive reagent to provide an increased benefit than if they were administered alone. For example, each (e.g., HSP preparation and immunoreactive reagent) may be administered at the same time or sequentially in any order at different points in time; however, if not administered at the same time, they should be administered sufficiently close in time so as to provide the desired therapeutic or prophylactic effect Each can be administered separately, in any appropriate form and by any suitable route. In one embodiment, the HSP preparation and the immunoreactive reagent are administered by the same mode of administration. In another embodiment, the HSP preparation and the immunoreactive reagent are administered by different routes of administration. The administration of each may be at the same or different sites, e.g., arm and leg.

In various embodiments, the prophylactic or therapeutic agents are administered less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In another embodiment, the prophylactic or therapeutic agents are administered at 1, 2, 4, 8, 12, 24, or 48 hours apart. In other embodiments, the HSP preparation and immunoreactive reagent are administered 2 to 4 days apart, 1 week apart, 1 to 2 weeks apart, 2 to 4 weeks apart, one month apart, 1 to 2 months apart, or 2 or more months apart. In preferred embodiments, two or more components are administered within the same patient visit. Preferably, the HSP preparation and the immunoreactive reagent are administered to a subject within a time frame that allows for both the HSP preparation and the immunoreactive reagent to both be active at the same time. One skilled in the art would be able to determine such a time frame by determining the half life of the HSP preparation and the immunoreactive reagent.

In a specific embodiment, the HSP preparation is administered prior to the administration of the immunoreactive reagent. In alternate specific embodiment, the HSP preparation is administered subsequent to the administration of the immunoreactive reagent.

In certain embodiments, the HSP preparation and/or immunoreactive reagent are cyclically administered to a subject. Cycling therapy involves the administration of the HSP preparation for a period of time, followed by the administration of an immunoreactive reagent and/or any third agent for a period of time and repeating this sequential administration. Cycling therapy reduces the development of resistance to one or more of the therapies, avoids or reduces the side effects of one of the therapies, and/or improves the efficacy of the treatment. In such embodiments, the invention contemplates the alternate administration of an HSP preparation followed by the administration of an immunoreactive reagent 4 to 6 days later, preferably 2 to 4 days later, more preferably 1 to 2 days later, followed by the administration of an HSP preparation 4 to 6 days later, preferably 2 to 4 days later, more preferably 1 to 2 days later, etc. Such a cycle may be repeated as many times as desired.

In other embodiments, the HSP preparation is administered to a subject at reasonably the same time as the immunoreactive reagent. Preferably, the two administrations are performed within a time frame of less than one minute to about five minutes, about five minutes to about 10 minutes, about 10 minutes to 30 minutes, about 30 minutes up to about sixty minutes from each other, for example, at the same doctor's visit.

In other embodiments, the immunoreactive reagent and the HSP preparation can be administered simultaneously. In certain embodiments, the immunoreactive reagent and an HSP preparation is administered as a single pharmaceutical composition. In such embodiments, a pharmaceutical composition of the invention is administered once a day, twice a day, or three times a day. In other embodiments, the pharmaceutical composition is administered once a week, twice a week, once every two weeks, once a month, once every six weeks, once every two months, twice a year or once per year. It will also be appreciated that the effective dosage of the immunoreactive reagents used for treatment may increase or decrease over the course of a particular treatment.

Another therapeutic method is also provided. In this embodiment, an HSP preparation is administered to a subject when it is desired that the APCs of the subject be in an activated state. The HSP preparation can be administered regularly for a period of time, e.g., daily for up to several weeks—1 to 2 weeks, 2 to 4 weeks, 4 to 6 weeks, up to two months—which may precede, overlap, and/or follow a treatment regimen with an immunoreactive reagent. The HSP preparation can improve the outcome of the treatment. Without being bound by any theory or mechanism, the administration of an HSP preparation to a subject can enhance the responsiveness of non-specific immune mechanisms of the subject, for example, by increasing the number of natural killer (NK) cells and/or accelerating the maturation of dendritic cells. In certain embodiments, the activation of APCs by the HSP preparations is ex vivo and such activated APCs are subsequently administered according to the methods and compositions of the invention. A HSP preparation that is the same as or different from the HSP preparation to be administered can be used for activating the APCs. Each HSP preparation may or may not display the immunogenicity of the antigenic molecule recognized by the immunoreactive reagent.

In another embodiment, the HSP preparation is administered to a subject within a time frame of one hour to twenty four hours after the administration of an immunoreactive reagent. The time frame can be extended if a slow- or continuous-release type of immunoreactive reagent is used. This method is believed to help activate effector cells, such as APCs present in at or near the site of administration that may not yet have been activated by the presence of the immunoreactive reagent.

In yet another embodiment, the HSP preparation is administered to a subject within a time frame of one to 12 hours, 12 to 24 hours, 24 to 48 hours before the administration of an immunoreactive reagent. This method is believed to pre-activate the subject's APCs prior to the encounter with the immunoreactive reagent.

In other embodiments, courses of treatment are administered concurrently, i.e., individual doses of the HSP preparation and the immunoreactive agent(s) are administered separately yet within a time interval such that the HSP preparation can work together with the immunoreactive reagent(s). For example, the HSP preparation may be administered one time per week in combination with the immunoreactive reagent(s) that may be administered one time every two weeks or one time every three weeks. In other words, the dosing regimens for the HSP preparation and immunoreactive reagent(s) are carried out simultaneously even if each is not administered simultaneously or within the same patient visit.

In one embodiment, an HSP preparation is administered concurrently with one or more immunoreactive reagents in the same pharmaceutical composition. In another embodiment, an HSP preparation is administered concurrently with one or more immunoreactive reagents in separate pharmaceutical compositions. In still another embodiment, an HSP preparation is administered prior to or subsequent to administration of one or more immunoreactive reagents. The invention contemplates administration of an an HSP preparation in combination with one or more immunoreactive reagents by the same or different routes of administration. In a preferred embodiment, the HSP preparation is administered intradermally. In another preferred embodiment, the immunoreactive agent is administered intravenously. In a particularly preferred embodiment, the HSP preparation is administered intradermally and the immunoreactive agent is administered intravenously. In certain embodiments, when an HSP preparation is administered concurrently with an immunoreactive reagent that potentially produces adverse or unwanted side effects including, but not limited to toxicity, said immunoreactive reagent can advantageously be administered at a dose that falls below the threshold that the adverse side effect is elicited.

In another embodiment, the invention provides for a method of inducing an immune response by a sub-optimal amount of an immunoreactive reagent, wherein the HSP preparation facilitates the induction of an immune response by an amount of an immunoreactive reagent which is otherwise insufficient for inducing the immune response when used alone. In certain embodiments, a sub-optimal amount is an amount otherwise insufficient to efficiently induce an immune response or the prophylactically or therapeutically desired effect. In particular, the method comprises the steps of: (a) administering to the subject an amount of a heat shock protein preparation; and (b) administering to the subject an immunoreactive reagent against which an immune response is desired to be induced in an amount that is sub-optimal in the absence of step (a), whereby an immune response is induced in the subject. The HSP preparation may or may not display the immunogenicity of the antigenic molecule recognized by the immunoreactive reagent.

In yet another embodiment, the invention provides for a method of inducing an immune response by a sub-immunogenic amount of an HSP preparation, wherein the immunoreactive reagent facilitates the induction of an immune response by an amount of an HSP preparation less efficient for inducing the immune response when used alone. The HSP preparation may or may not display the immunogenicity of the antigenic molecule recognized by the immunoreactive reagent.

The invention provides methods of treatment, prevention, and amelioration of one or more symptoms associated with a disease, disorder or infection by administering to a subject a pharmaceutical composition comprising an immunoreactive reagent and an HSP. In a preferred aspect, the immunoreactive reagent and HSP are substantially purified (i.e., substantially free from substances that limit its effect or produce undesired side-effects). In accordance with the present invention, a composition of the invention, comprising an immunoreactive reagent and an HSP is administered to a human subject with cancer, an infectious disease, or a neurodegenerative or amyloid diseases as a treatment.

The present invention also relates to methods of using the compositions of the invention for the treatment of infectious diseases, primary and metastatic neoplastic diseases (i.e., cancer), neurodegenerative or amyloid diseases, protein deposition/amyloidogenic diseases or any other treatment of a disease that would be improved by an enhanced immune response.

### 4.2 Patient Population

The subject to which the HSP preparation and immunoreactive reagent are administered is preferably a mammal such as a non-primate (e.g., cows, pigs, horses, cats, dogs, rats etc.) and a primate (e.g., monkey such as a cynomolgous monkey and a human). In a preferred embodiment, the subject is a human.

In other various embodiments, the methods and compositions of the invention are used to treat or prevent any disease or disorder in which a therapeutic or prophylactic immunoreactive reagent is useful for treatment or prophylaxis. Preferably the disease or disorder is amenable to treatment or prevention by an enhanced immune response, more preferably an infectious disease, cancer or a neurodegenerative or amyloid disorder.

The compositions can be utilized for the prevention of a variety of cancers, e.g., in individuals who are predisposed as a result of familial history or in individuals with an enhanced risk to cancer due to environmental factors, for the prevention of infectious diseases, e.g., in individuals with enhanced risks of exposure to agents of infectious disease, and for the prevention of neurodegenerative or amyloid diseases, for example in individuals with genetic predispositions to neurodegenerative or amyloid diseases.

The methods and compositions of the invention may be used in patients who are treatment naive, in patients who have previously received or are currently receiving treatment with an HSP preparation, in patients who have previously received or are currently receiving treatment with an immunoreactive reagent, or in patients who have previously received or are currently receiving treatment with other pharmaceutical agents or combinations, including but not limited to anti-cancer agents, antibiotics, anti-bacterial agents, anti-fungal agents and anti-viral agents. In a specific embodiment of the invention, an HSP preparation is administered to a patient that has previously received or is currently receiving treatment with immunotherapeutic reagents. In another embodiment, an immunotherapeutic reagent is administered to a patient that has previously received or is currently receiving treatment with an HSP preparation. In yet another embodiment of the invention, an HSP preparation is administered to a patient that has previously received or is currently receiving treatment that includes, but is not limited to, anti-cancer agents, antibiotics, anti-bacterial agents, anti-fungal agents or anti-viral agents, optionally with an immunoreactive reagent. In still another embodiment, an immunotherapeutic reagent is administered to a patient that has previously received or is currently receiving treatment that includes, but is not limited to, anti-cancer agents, antibiotics, anti-bacterial agents, anti-fungal agents or anti-viral agents, optionally with an HSP preparation.

In a preferred embodiment, a pharmaceutical composition of the invention consisting of an immunotherapeutic reagent and an HSP preparation is administered to a patient that has previously received or is currently receiving treatment that includes, but is not limited to, anti-cancer agents, antibiotics, anti-bacterial agents, anti-fungal agents or anti-viral agents.

The methods and compounds of the invention may also be used to treat patients that have previously received treatment with HSP preparations or immunoreactive reagents and are currently not efficiently treated with respect to each treatment administered alone.

In one embodiment, a composition of the invention consisting of an HSP preparation and an immunoreactive reagent is administered to a patient not sufficiently susceptible to single-agent treatment with an HSP preparation alone. In another embodiment, a composition of the invention consisting of an HSP preparation and an immunoreactive reagent is administered to a patient that is refractory to single-agent treatment with an immunoreactive reagent alone. In yet another embodiment, a composition of the invention consisting of an HSP preparation and an immunoreactive reagent is administered to a patient that is refractory to treatment with both an HSP preparation alone and an immunoreactive reagent alone, but not together. In still another embodiment, a composition of the invention consisting of an HSP preparation and an immunoreactive reagent is administered to a patient that is not receiving any form of medical treatment.

### 4.3 Treatment and Prevention of Cancer

The invention encompasses methods for treating or preventing a cancer or metastasis in a subject comprising in any order the steps of administering to the subject an immunoreactive reagent comprising a component that recognizes the antigen or epitope of a cancer cell (e.g., an immunogenic amount of an antigen on a cancer, such as but not limited to a tumor-specific antigen, and a tumor-associated antigen, or a molecule displaying antigenicity thereof); and administering to the subject an amount of an HSP preparation effective to induce or increase an immune response in the subject to the component recognized by the immunoreactive reagent.

In certain embodiments, the compositions and methods of the invention can be used to prevent, inhibit or reduce the growth or metastasis of cancerous cells. In a specific embodiment, the administration of an HSP preparation in combination with an immunoreactive reagent inhibits or reduces the growth or metastasis of cancerous cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the growth or metastasis in absence of the administration of said HSP preparation in combination with said immunoreactive reagent.

Cancers that can be treated according to the methods of the invention include, but are not limited to, leukemia (e.g., acute leukemia such as acute lymphocytic leukemia and acute myelocytic leukemia), neoplasms, tumors (e.g., non-Hodgkin's lymphoma, fibrosarcoma, myxosarcoma, liposarcoma, chondrosarcoma, osteogenic sarcoma, chordoma, angiosarcoma, endothehosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma), heavy chain disease (B-cell lymphoma), metastases, or any disease or disorder characterized by uncontrolled cell growth.

Tumor antigens or tumor associated antigens include cancer-germ cell (CG) antigens (MAGE, NY-ESO-1), mutational antigens (MUM-1, p53, CDK-4), over-expressed self-antigens (p53, HER2/NEU), viral antigens (from Papilloma Virus, Epstein-Barr Virus), tumor proteins derived from non-primary open reading frame mRNA sequences (Y-ESO1, LAGE1), Melan A, MART-1, MAGE-1, MAGE-3, BAGE, GAGE-1, GAGE-2, tyrosinase, gp100, gp75, HER-2/neu, c-erb-B2, CEA, PSA, MUC-1, CA-125, Stn, TAG-72, KSA (17-1A), PSMA, p53 (point mutated and/or overexpressed), RAS (point mutated), EGF-R, VEGF, GD2, GM2, GD3, Anti-Id, CD20, CD19, CD22, CD36, Aberrant class II, B1, CD25 (IL-2R) (anti-TAC), or HPV.

In a preferred embodiment, a method or composition of the invention is used for treating or preventing a cancer or metastasis in a subject comprising the administration of an HSP preparation and an immunoreactive reagent where the immunoreactive reagent is an anti-CTLA-4 antibody or an anti-41BB antibody. In another preferred embodiment, a method or composition of the invention is used for treating or preventing a cancer or metastasis in a subject comprising the administration of an HSP preparation and an immunoreactive reagent where the immunoreactive reagent is an anti-tumor monoclonal antibody. In yet another preferred embodiment, a method or composition of the invention is used for treating or preventing a cancer or metastasis in a subject comprising the administration of an HSP preparation and an immunoreactive reagent where the immunoreactive reagent is Herceptin.

### 4.4 Treatment of Infectious Diseases

The invention also encompasses methods for treating or preventing an infectious disease in a subject comprising in any order the steps of administering to the subject an immunoreactive reagent; and administering to the subject an amount of a heat shock protein preparation effective in combination with the immunoreactive reagent to induce or increase an immune response to the component in the subject.

Infectious diseases that can be treated or prevented by use of an immunoreactive reagent in conjunction with the methods of the present invention are caused by infectious agents including, but not limited to, viruses, bacteria, fungi protozoa and parasites. Some of the commonly-used immunoreactive reagents against infectious diseases and their appropriate doses and uses are known in the art and described in literature such as the Physician's Desk Reference (56^{t} ed., 2002).

Infectious agents that can be treated according to the invention include, but are not limited to viruses, bacteria, fungi, and agents of protozoal disease.

Viral diseases that can be treated or prevented by use of an immunoreactive reagent in conjunction with the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, small pox, Epstein Barr virus, human immunodeficiency virus type I (HIV-I), human immunodeficiency virus type II (HIV-II), and agents of viral diseases such as viral miningitis, encephalitis, dengue or small pox.

Bacterial diseases that can be treated or prevented by use of an immunoreactive reagent in conjunction with the methods of the present invention are caused by bacteria including, but not limited to, mycobacteria rickettsia, mycoplasma, neisseria, S. pneumonia, Borrelia burgdorferi (Lyme disease), Bacillus antracis (anthrax), tetanus, streptococcus, staphylococcus, mycobacterium, tetanus, pertissus, cholera, plague, diptheria, chlamydia, S. aureus and legionella.

Protozoal diseases and/or parasitic diseases that can be treated or prevented by use of an immunoreactive reagent in conjunction with the methods of the present invention are caused by protozoa and/or parasites including, but not limited to, leishmania, kokzidioa, trypanosoma, malaria, chlamydia, rickettsia, Chagas' disease, filiariasis, toxoplasmosis, schistosomiasis, and diseases caused by tapeworms.

### 4.5 Treatment of Neurodegenerative Diseases

Immunoreactive reagents specifically binding an antigenic molecule in or on a cell or structure, e.g., extracellular deposits or plaques comprising peptide and/or protein fibrils, that displays the hallmarks of a neurodegenerative or amyloid disease may also be utilized. Preferably, where it is desired to treat or prevent neurodegenerative or amyloid diseases, immunoreactive reagents that specifically bind to molecules comprising epitopes of antigenic molecules associated with neurodegenerative diseases, or epitopes of antigenic molecules associated with amyloid diseases, including but not limited to fibril peptides or proteins, are used. Such neurodegenerative disease-associated antigenic molecules may be molecules associated with Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, prion diseases, spongiform encephalopathies, Creutzfeldt-Jakob disease, polyglutamine diseases, Huntington's disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorder, hypertension, neuropsychiatric disorder, schizophrenia, or schizoaffective disorder.

Examples of such antigenic molecules are disclosed in WO 01/52890 published Jul. 26, 2001, which is incorporated by reference herein in its entirety, and include, but are not limited to, β-amyloid or a fragment thereof, an oligomeric Aβ complex or a fragment thereof, an ApoE4-Aβ complex, tau protein or a fragment thereof, amyloid precursor protein or a fragment thereof, a mutant amyloid precursor protein or a fragment thereof, presenillin or a fragment thereof, a mutant of presenillin or a fragment thereof, α-synuclein or a fragment thereof, or a prion protein or a fragment thereof, and the antigenic derivatives of any of the foregoing proteins or fragments thereof. Amyloid disease associated antigenic molecules may be molecules associated with diseases characterized by the extracellular deposition of protein and/or peptide fibrils which form amyloid deposits or plaques, including but not limited to type II diabetes and amyloidoses associated with chronic inflammatory or infectious disease states and malignant neoplasms, e.g., myeloma. Certain amyloid disease such as but not limited to Alzheimer's disease and prion diseases, e.g., Creutzfeldt Jacob disease, are also neurodegenerative diseases.

### 4.6 HSP Preparations

Any HSP or HSP preparation known in the art may be used in the compositions and methods of the invention. For the purposes of this invention, HSP preparations can include, but are not limited to, free HSP not bound to any molecule, molecular complexes of HSP with another molecule, such as a peptide, and HSP fusion proteins. An HSP-peptide complex comprises an HSP covalently or noncovalently attached to a peptide. The HSP-peptide complex may consist of HSPs bound to peptides derived from the tumor, pathogen or cell type and/or protein of interest (e.g., same target as is recognized by the antibody). Alternately, the HSP-peptide complex may consist of HSPs bound to an endogenous peptide, but not necessarily a peptide from the same source as the target of the therapeutic antibody. The methods of the invention do not require covalent or noncovalent attachment to any specific antigens or antigenic peptides prior to administration to a subject. The HSP preparation may or may not be obtained from the subject the preparation is administered to. The HSP, HSP-peptide complex, or HSP fusion protein is preferably purified. An HSP preparation may include crude cell lysate comprising HSP, the amount of lysate corresponding to between 100 to 10⁸ cell equivalents. When a peptide is attached to an HSP, the peptide may be any peptide, which can be noncovalently, covalently bound, or fused to the HSP. HSPs can be conveniently purified from most cellular sources as a population of complexes of different peptides non-covalently bound to HSPs. The HSPs can be separated from the non-covalently bound peptides by exposure to low pH and/or adenosine triphosphate, or other methods known in the art. Generally, the HSP preparation is separately administered from the immunoreactive reagent. The peptide(s) may be unrelated to the immunoreactive reagent, or the infectious disease or disorder in question. For convenience and comfort of a recipient, the HSP preparation can be mixed with the immunoreactive reagent immediately prior to administration.

In various embodiments, the source of the HSP is preferably an eukaryote, more preferably a mammal, and most preferably a human. Accordingly, the HSP preparation used by the methods of the invention includes eukaryotic HSPs, mammalian HSPs and human HSPs. The eukaryotic source from which the HSP preparation is derived and the subject receiving the HSP preparation are preferably the same species.

In various embodiments of the invention, the HSP preparation may comprise HSPs including but not limited to, hsp60, hsp70, hsp90, hsp110, gp96, grp170 or calreticulin, singly or in combination with each other. Preferably, the HSP is hsp60, hsp70, hsp90, hsp110, gp96, grp170, or calreticulin. Also encompassed by the invention are HSP-peptide complexes such as hsp60- peptide complexes, hsp70-peptide complexes, hsp90-peptide complexes, hsp110-peptide complexes, gp96-peptide complexes, grp170-peptide complexes or calreticulin-peptide complexes. Also encompassed by the invention are HSP fusion proteins such as hsp60 fusion proteins, hsp70 fusion proteins, hsp90 fusion proteins, hsp110 fusion proteins, gp96 fusion proteins, grp170 fusion proteins or calreticulin fusion proteins.

In a preferred embodiment, the HSP preparation comprises a single HSP, HSP complex, or HSP fusion protein. In other embodiments of the invention, an HSP preparation comprises mixtures of HSPs, HSP complexes, or HSP fusion proteins. Preferably, the mixture of HSPs, HSP complexes, and/or HSP fusion proteins comprises two or more substantially pure HSPs, HSP complexes, and/or HSP fusion proteins. As used herein, “substantially pure” means substantially free from compounds normally associated with the HSP or HSP complex in its natural state and exhibiting constant and reproducible chromatographic response, elution profiles, and biologic activity. Substantially pure HSP complexes are not stripped of the peptides that are covalently or non-covalently complexed to the HSP or the peptides that are endogenously complexed to the HSP. The term “substantially pure” is not meant to exclude artificial or synthetic mixtures of the HSP, HSP complex, or HSP fusion proteins with other compounds. A number of non-limiting examples of HSPs, HSP complexes, and HSP fusion proteins and their methods of preparation are provided below.

In one embodiment, when the HSP preparation is not used in conjunction with an immunoreactive reagent to elicit a specific immune response, administering the HSP preparation alone does not induce the antigen-specific immune response that would have been induced by the immunoreactive reagent. In another embodiment, the HSP preparation does induce the antigen-specific immune response that would have been induced by the immunoreactive reagent.

It is contemplated that all HSPs belonging to the hsp60, hsp70 and hsp90 families, including fragments of such HSPs, can be used in the practice of the instant invention.

In the present invention, purified unbound HSPs, HSPs covalently or noncovalently bound to specific peptides or nonspecific peptides (collectively referred to herein as HSP-peptide complexes), HSP fusion proteins, and combinations thereof are used. Purification of HSPs in complexed or non-complexed forms are described in the following subsections. Further, one skilled in the art can synthesize HSPs and HSP fusion proteins by recombinant expression or peptide synthesis, which are also described below.

In another embodiment, it is contemplated that the HSPs can be other proteins, muteins, analogs, and variants thereof having at least 35% to 55%, preferably 55% to 75%, and most preferably 75% to 85% amino acid identity with members of the three major families of stress proteins whose expression levels in a cell are enhanced in response to a stressful stimulus. The preparation, isolation, and purification of stress proteins belonging to the HSP class are known in the art and described in literature, for example the preparation and purification of calreticulin is described in Basu and Srivastava, 1999 J. Expt. Med. 189:797-802, herein incorporated by reference in its entirety. The invention also encompasses methods for preparing and purifying HSPs and HSP-peptide complexes and are described below and presented by way of example not by way of limitation.

### 4.6.1 Preparation and Purification of Hsp70 or Hsp70-Peptide Complexes

The purification of hsp70-peptide complexes has been described previously, see, for example, Udono et al., 1993, J. Exp. Med. 178:1391-1396. A procedure that may be used, presented by way of example but not limitation, is as follows:

Initially, human or mammalian cells are suspended in 3 volumes of 1× Lysis buffer consisting of 5 mM sodium phosphate buffer (pH 7), 150 mM NaCl, 2 mM CaCl₂, 2 mM MgCl₂ and 1 mM phenyl methyl sulfonyl fluoride (PMSF). Then, the pellet is sonicated, on ice, until >99% cells are lysed as determined by microscopic examination. As an alternative to sonication, the cells may be lysed by mechanical shearing and in this approach the cells typically are resuspended in 30 mM sodium bicarbonate (pH 7.5), 1 mM PMSF, incubated on ice for 20 minutes and then homogenized in a Dounce homogenizer until >95% cells are lysed.

Then the lysate is centrifuged at 1,000 g for 10 minutes to remove unbroken cells, nuclei and other cellular debris. The resulting supernatant is recentrifuged at 100,000 g for 90 minutes, the supernatant harvested and then mixed with Con A Sepharose™ equilibrated with phosphate buffered saline (PBS) containing 2 mM Ca²⁺ and 2 mM Mg²⁺. When the cells are lysed by mechanical shearing the supernatant is diluted with an equal volume of 2× lysis buffer prior to mixing with Con A Sepharose™. The supernatant is then allowed to bind to the Con A Sepharose™ for 2-3 hours at 4° C. The material that fails to bind is harvested and dialyzed for 36 hours (three times, 100 volumes each time) against 10 mM Tris-Acetate (pH 7.5), 0.1 mM EDTA, 10 mM NaCl, 1 mM PMSF. Then the dialyzate is centrifuged at 17,000 rpm (Sorvall SS34 rotor) for 20 minutes. Then the resulting supernatant is harvested and applied to a Mono Q FPLC™ ion exchange chromatographic column (Pharmacia) equilibrated in 20 mM Tris-Acetate (pH 7.5), 20 mM NaCl, 0.1 mM EDTA and 15 mM 2-mercaptoethanol. The column is then developed with a 20 mM to 50 mM NaCl gradient and then eluted fractions fractionated by sodium dodecyl sulfate-polyacrylamide gel electrophoresis (SDS-PAGE) and characterized by immunoblotting using an appropriate anti-hsp70 antibody (such as from clone N27F3-4, from StressGen).

Fractions strongly immunoreactive with the anti-hsp70 antibody are pooled and the hsp70-peptide complexes precipitated with ammonium sulfate; specifically with a 50%-70% ammonium sulfate cut. The resulting precipitate is then harvested by centrifugation at 17,000 rpm (SS34 Sorvall rotor) and washed with 70% ammonium sulfate. The washed precipitate is then solubilized and any residual ammonium sulfate removed by gel filtration on a Sephadex^{R} G25 column (Pharmacia). If necessary the hsp70 preparation thus obtained can be repurified through the Mono Q FPLC™ ion exchange chromatographic column (Pharmacia) as described above.

The hsp70-peptide complex can be purified to apparent homogeneity using this method. Typically 1 mg of hsp70-peptide complex can be purified from 1 g of cells/tissue.

An improved method for purification of hsp70-peptide complexes comprises contacting cellular proteins with ADP or a nonhydrolyzable analog of ATP affixed to a solid substrate, such that hsp70 in the lysate can bind to the ADP or nonhydrolyzable ATP analog, and eluting the bound hsp70. A preferred method uses column chromatography with ADP affixed to a solid substratum (e.g., ADP-agarose). The resulting hsp70 preparations are higher in purity and devoid of contaminating proteins that are not the endogenously bound peptides associated with the HSP in an HSP-peptide complex. The hsp70 complex yields are also increased significantly by about more than 10 fold. Alternatively, chromatography with nonhydrolyzable analogs of ATP, instead of ADP, can be used for purification of hsp70-peptide complexes. By way of example but not imitation, purification of hsp70-peptide complexes by ADP-agarose chromatography can be carried out as follows:

Meth A sarcoma cells (500 million cells) are homogenized in hypotonic buffer and the lysate is centrifuged at 100,000 g for 90 minutes at 4° C. The supernatant is applied to an ADP-agarose column. The column is washed in buffer and is eluted with 5 column volumes of 3 mM ADP. The hsp70-peptide complexes elute in fractions 2 through 10 of the total 15 fractions which elute. The eluted fractions are analyzed by SDS-PAGE. The hsp70-peptide complexes can be purified to apparent homogeneity using this procedure.

Separation of the HSP from an hsp70-peptide complex can be performed in the presence of ATP or low pH. These two methods may be used to elute the peptide from an hsp70-peptide complex. The first approach involves incubating an hsp70-peptide complex preparation in the presence of ATP. The other approach involves incubating an hsp70-peptide complex preparation in a low pH buffer. These methods and any others known in the art may be applied to separate the HSP and peptide from an hsp-peptide complex.

### 4.6.2 Preparation and Purification of Hsp90 or Hsp90-Peptide Complexes

A procedure that can be used, presented by way of example and not limitation, is as follows:

Initially, human or mammalian cells are suspended in 3 volumes of 1× Lysis buffer consisting of 5 mM sodium phosphate buffer (pH 7), 150 mM NaCl, 2 mM CaCl₂, 2 mM MgCl₂ and 1 mM phenyl methyl sulfonyl fluoride (PMSF). Then, the pellet is sonicated, on ice, until >99% cells are lysed as determined by microscopic examination. As an alternative to sonication, the cells may be lysed by mechanical shearing and in this approach the cells typically are resuspended in 30 mM sodium bicarbonate (pH 7.5), 1 mM PMSF, incubated on ice for 20 minutes and then homogenized in a Dounce homogenizer until >95% cells are lysed.

Then the lysate is centrifuged at 1,000 g for 10 minutes to remove unbroken cells, nuclei and other cellular debris. The resulting supernatant is recentrifuged at 100,000 g for 90 minutes, the supernatant harvested and then mixed with Con A Sepharose™ equilibrated with PBS containing 2 mM Ca²⁺ and 2 mM Mg²⁺. When the cells are lysed by mechanical shearing the supernatant is diluted with an equal volume of 2× Lysis buffer prior to mixing with Con A Sepharose™. The supernatant is then allowed to bind to the Con A Sepharose™ for 2-3 hours at 4° C. The material that fails to bind is harvested and dialyzed for 36 hours (three times, 100 volumes each time) against 10 mM Tris-Acetate (pH 7.5), 0.1 mM EDTA, 10 mM NaCl, 1 mM PMSF. Then the dialyzate is centrifuged at 17,000 rpm (Sorvall SS34 rotor) for 20 minutes. Then the resulting supernatant is harvested and applied to a Mono Q FPLC™ ion exchange chromatographic column (Pharmacia) equilibrated with lysis buffer. The proteins are then eluted with a salt gradient of 200 mM to 600 mM NaCl.

The eluted fractions are fractionated by SDS-PAGE and fractions containing the hsp90-peptide complexes identified by immunoblotting using an anti-hsp90 antibody such as 3G3 (Affinity Bioreagents). Hsp90-peptide complexes can be purified to apparent homogeneity using this procedure. Typically, 150-200 μg of hsp90-peptide complex can be purified from 1 g of cells/tissue.

Separation of the HSP from an hsp90-peptide complex can be performed in the presence of ATP or low pH. These two methods may be used to elute the peptide from an hsp90-peptide complex. The first approach involves incubating an hsp90-peptide complex preparation in the presence of ATP. The other approach involves incubating an hsp90-peptide complex preparation in a low pH buffer. These methods and any others known in the art may be applied to separate the HSP and peptide from an hsp-peptide complex.

### 4.6.3 Preparation and Purification of Gp96 or Gp96-Peptide Complexes

A procedure that can be used, presented by way of example and not limitation, is as follows:

A pellet of human or mammalian cells is resuspended in 3 volumes of buffer consisting of 30 mM sodium bicarbonate buffer (pH 7.5) and 1 nM PMSF and the cells allowed to swell on ice 20 minutes. The cell pellet is then homogenized in a Dounce homogenizer (the appropriate clearance of the homogenizer will vary according to each cell type) on ice until >95% cells are lysed.

The lysate is centrifuged at 1,000 for 10 minutes to remove unbroken cells, nuclei and other debris. The supernatant from this centrifugation step is then recentrifuged at 100,000 g for 90 minutes. The gp96-peptide complex can be purified either from the 100,000 pellet or from the supernatant.

When purified from the supernatant, the supernatant is diluted with equal volume of 2× lysis buffer and the supernatant mixed for 2-3 hours at 4° C. with Con A Sepharose™ equilibrated with PBS containing 2 mM Ca²⁺ and 2 mM Mg²⁺. Then, the slurry is packed into a column and washed with 1× lysis buffer until the OD₂₈₀ drops to baseline. Then, the column is washed with ⅓ column bed volume of 10% α-methyl mannoside (α-MM) dissolved in PBS containing 2 mM Ca²⁺ and 2 mM Mg²⁺, the column sealed with a piece of parafilm, and incubated at 37° C. for 15 minutes. Then the column is cooled to room temperature and the parafilm removed from the bottom of the column. Five column volumes of the α-MM buffer are applied to the column and the eluate analyzed by SDS-PAGE. Typically the resulting material is about 60-95% pure, however this depends upon the cell type and the tissue-to-lysis buffer ratio used. Then the sample is applied to a Mono Q FPLC™ ion exchange chromatographic column (Pharmacia) equilibrated with a buffer containing 5 mM sodium phosphate (pH 7). The proteins are then eluted from the column with a 0-1M NaCl gradient and the gp96 fraction elutes between 400 mM and 550 mM NaCl.

The procedure, however, may be modified by two additional steps, used either alone or in combination, to consistently produce apparently homogeneous gp96-peptide complexes. One optional step involves an ammonium sulfate precipitation prior to the Con A purification step and the other optional step involves DEAE-Sepharose™ purification after the Con A purification step but before the Mono Q FPLC™ step.

In the first optional step, described by way of example as follows, the supernatant resulting from the 100,000g centrifugation step is brought to a final concentration of 50% ammonium sulfate by the addition of ammonium sulfate. The ammonium sulfate is added slowly while gently stirring the solution in a beaker placed in a tray of ice water. The solution is stirred from about ½ to 12 hours at 4° C. and the resulting solution centrifuged at 6,000 rpm (Sorvall SS34 rotor). The supernatant resulting from this step is removed, brought to 70% ammonium sulfate saturation by the addition of ammonium sulfate solution, and centrifuged at 6,000 rpm (Sorvall SS34 rotor). The resulting pellet from this step is harvested and suspended in PBS containing 70% ammonium sulfate in order to rinse the pellet. This mixture is centrifuged at 6,000 rpm (Sorvall SS34 rotor) and the pellet dissolved in PBS containing 2 mM Ca²⁺ and Mg²⁺. Undissolved material is removed by a brief centrifugation at 15,000 rpm (Sorvall SS34 rotor). Then, the solution is mixed with Con A Sepharose™ and the procedure followed as before.

In the second optional step, described by way of example as follows, the gp96 containing fractions eluted from the Con A column are pooled and the buffer exchanged for 5 mM sodium phosphate buffer (pH 7), 300 mM NaCl by dialysis, or preferably by buffer exchange on a Sephadex G25 column. After buffer exchange, the solution is mixed with DEAE-Sepharose™ previously equilibrated with 5 mM sodium phosphate buffer (pH 7), 300 mM NaCl. The protein solution and the beads are mixed gently for 1 hour and poured into a column. Then, the column is washed with 5 mM sodium phosphate buffer (pH 7), 300 mM NaCl, until the absorbance at 280 nm drops to baseline. Then, the bound protein is eluted from the column with five volumes of 5 mM sodium phosphate buffer (pH 7), 700 mM NaCl. Protein containing fractions are pooled and diluted with 5 mM sodium phosphate buffer (pH 7) in order to lower the salt concentration to 175 mM. The resulting material then is applied to the Mono Q FPLC™ ion exchange chromatographic column (Pharmacia) equilibrated with 5 mM sodium phosphate buffer (pH 7) and the protein that binds to the Mono Q FPLC™ ion exchange chromatographic column (Pharmacia) is eluted as described before.

It is appreciated, however, that one skilled in the art may assess, by routine experimentation, the benefit of incorporating the second optional step into the purification protocol. In addition, it is appreciated also that the benefit of adding each of the optional steps will depend upon the source of the starting material.

When the gp96 fraction is isolated from the 100,000 g pellet, the pellet is suspended in 5 volumes of PBS containing either 1% sodium deoxycholate or 1% oxtyl glucopyranoside (but without the Mg²⁺ and Ca²⁺) and incubated on ice for 1 hour. The suspension is centrifuged at 20,000 g for 30 minutes and the resulting supernatant dialyzed against several changes of PBS (also without the Mg²⁺ and Ca²⁺) to remove the detergent. The dialysate is centrifuged at 100,000 g for 90 minutes, the supernatant harvested, and calcium and magnesium are added to the supernatant to give final concentrations of 2 mM, respectively. Then the sample is purified by either the unmodified or the modified method for isolating gp96-peptide complex from the 100,000 g supernatant, see above.

The gp96-peptide complexes can be purified to apparent homogeneity using this procedure. About 10-20 μg of gp96 can be isolated from 1 g cells/tissue.

Separation of the HSP from an gp96-peptide complex can be performed in the presence of ATP or low pH. These two methods may be used to elute the peptide from an gp96-peptide complex. The first approach involves incubating an gp96-peptide complex preparation in the presence of ATP. The other approach involves incubating an gp96-peptide complex preparation in a low pH buffer. These methods and any others known in the art may be applied to separate the HSP and peptide from an hsp-peptide complex.

### 4.6.4 Preparation and Purification of HsP110-Peptide Complexes

A procedure, described by Wang et al., 2001, J. Immunol. 166(1):490-7, that can be used, presented by way of example and not limitation, is as follows:

A pellet (40-60 ml) of cell or tissue, e.g., tumor cell tissue, is homogenized in 5 vol of hypotonic buffer (30 mN sodium bicarbonate, pH 7.2, and protease inhibitors) by Dounce homogenization. The lysate is centrifuged at 4,500×g and then 100,000×g for 2 hours. If the cells or tissues are of hepatic origin, the resulting supernatant is was first applied to a blue Sepharose column (Pharmacia) to remove albumin. Otherwise, the resulting supernatant is applied to a Con A-Sepharose column (Pharmacia Biotech, Piscataway, N.J.) previously equilibrated with binding buffer (20 mM Tris-HCI, pH 7.5; 10 nM NaCl; 1 mM MgCl₂; 1 mM CaCl₂; 1 mM MnCl₂; and 15 mM 2-ME). The bound proteins are eluted with binding buffer containing 15% α-D-o-methylmannoside (Sigma, St. Louis, Mo.).

Con A-Sepharose unbound material is first dialyzed against a solution of 20 mM Tris-HCl, pH 7.5; 100 mM NaCl; and 15 mM 2-ME, and then applied to a DEAE-Sepharose column and eluted by salt gradient from 100 to 500 mM NaCl. Fractions containing hsp110 are collected, dialyzed, and loaded onto a Mono Q (Pharmacia) 10/10 column equilibrated with 20 mM Tris-HCl, pH 7.5; 200 mM NaCl; and 15 mM 2-ME. The bound proteins are eluted with a 200-500 mM NaCl gradient. Fractions are analyzed by SDS-PAGE followed by immunoblotting with an Ab for hsp110, as described by Wang et al., 1999, J. Immunol. 162:3378, Pooled fractions containing hsp110 are concentrated by Centriplus (Amicon, Beverly, Mass.) and applied to a Superose 12 column (Pharmacia). Proteins are eluted by 40 mM Tris-HCl, pH 8.0; 150 mM NaCl; and 15 mM 2-ME with a flow rate of 0.2 ml/min.

### 4.6.5 Preparation and Purification of Produced Grp170-Peptide Complexes

A procedure, described by Wang et al., 2001, J. Immunol. 166(1):490-7, that can be used, presented by way of example and not limitation, is as follows:

A pellet (40-60 ml) of cell or tissue, e.g., tumor cell tissue, is homogenized in 5 vol of hypotonic buffer (30 mN sodium bicarbonate, pH 7.2, and protease inhibitors) by Dounce homogenization. The lysate is centrifuged at 4,500×g and then 100,000×g for 2 hours. If the cells or tissues are of hepatic origin, the resulting supernatant is was first applied to a blue Sepharose column (Pharmacia) to remove albumin. Otherwise, the resulting supernatant is applied to a Con A-Sepharose column (Pharmacia Biotech, Piscataway, N.J.) previously equilibrated with binding buffer (20 mM Tris-HCI, pH 7.5; 100 mM NaCl; 1 mM MgCl₂; 1 mM CaCl₂; 1 mM MnCl₂; and 15 mM 2-ME). The bound proteins are eluted with binding buffer containing 15% α-D-o-methylmannoside (Sigma, St. Louis, Mo.).

Con A-Sepharose-bound material is first dialyzed against 20 mM Tris-HCl, pH 7.5, and 150 mM NaCl and then applied to a Mono Q column and eluted by a 150 to 400 mM NaCl gradient. Pooled fractions are concentrated and applied on the Superose 12 column (Pharmacia). Fractions containing homogeneous grp170 are collected.

### 4.6.6 Recombinant Expression of HSPs

Methods known in the art can be utilized to recombinantly produce HSPs. A nucleic acid sequence encoding an HSP can be inserted into an expression vector for propagation and expression in host cells.

An expression construct, as used herein, refers to a nucleotide sequence encoding an HSP operably associated with one or more regulatory regions which enables expression of the HSP in an appropriate host cell. “Operably-associated” refers to an association in which the regulatory regions and the HSP sequence to be expressed are joined and positioned in such a way as to permit transcription, and ultimately, translation.

The regulatory regions necessary for transcription of the HSP can be provided by the expression vector. A translation initiation codon (ATG) may also be provided if the HSP gene sequence lacking its cognate initiation codon is to be expressed. In a compatible host-construct system, cellular transcriptional factors, such as RNA polymerase, will bind to the regulatory regions on the expression construct to effect transcription of the modified HSP sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to the HSP gene sequence or to insert the HSP gene sequence into the cloning site of a vector, linkers or adapters providing the appropriate compatible restriction sites may be ligated to the ends of the cDNAs by techniques well known in the art (Wu et al., 1987, Methods in Enzymol. 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

An expression construct comprising an HSP sequence operably associated with regulatory regions can be directly introduced into appropriate host cells for expression and production of HSP-peptide complexes without further cloning. See, for example, U.S. Pat. No. 5,580,859. The expression constructs can also contain DNA sequences that facilitate integration of the HSP sequence into the genome of the host cell, e.g., via homologous recombination. In this instance, it is not necessary to employ an expression vector comprising a replication origin suitable for appropriate host cells in order to propagate and express the HSP in the host cells.

A variety of expression vectors may be used including, but not limited to, plasmids, cosmids, phage, phagemids or modified viruses; Typically, such expression vectors comprise a functional origin of replication for propagation of the vector in an appropriate host cell, one or more restriction endonuclease sites for insertion of the HSP gene sequence, and one or more selection markers. The expression vector must be used with a compatible host cell which may be derived from a prokaryotic or an eukaryotic organism including but not limited to bacteria, yeasts, insects, mammals and humans.

For long term, high yield production of properly processed HSP or HSP-peptide complexes, stable expression in mammalian cells is preferred. Cell lines that stably express HSP or HSP-peptide complexes may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while HSP is expressed continuously.

The recombinant cells may be cultured under standard conditions of temperature, incubation time, optical density and media composition. However, conditions for growth of recombinant cells may be different from those for expression of HSPs and antigenic proteins. Modified culture conditions and media may also be used to enhance production of the HSP. For example, recombinant cells containing HSPs with their cognate promoters may be exposed to heat or other environmental stress, or chemical stress. Any techniques known in the art may be applied to establish the optimal conditions for producing HSP or HSP-peptide complexes.

### 4.6.6.1 Recombinant Expression of HSP Fusion Proteins

Methods known in the art can be utilized to recombinantly produce fusion proteins comprised of a heat shock protein sequence and an antigenic peptide sequence. To produce such a recombinant fusion protein, an expression vector is constructed using nucleic acid sequences encoding a heat shock protein fused to sequences encoding an antigenic peptide, using recombinant methods known in the art, such as those described in Section 4.6.6, above. HSP-antigenic peptide fusions are then expressed and isolated. Such fusion proteins can be used to elicit an immune response. Suzue et al., 1997, Proc. Natl. Acad. Sci. U.S.A. 94:13146-51. By specifically designing the antigenic peptide portion of the molecule, such fusion proteins can be used to elicit an immune response and in immunotherapy against target diseases or disorders.

### 4.6.7 Peptide Synthesis

An alternative to producing HSP by recombinant techniques is peptide synthesis. For example, an entire HSP, or a peptide corresponding to a portion of an HSP can be synthesized by use of a peptide synthesizer. Conventional peptide synthesis or other synthetic protocols well known in the art may be used.

Peptides having the amino acid sequence of an HSP or a portion thereof may be synthesized by solid-phase peptide synthesis using procedures similar to those described by Merrifield, 1963, J. Am. Chem. Soc. 85:2149. During synthesis, N-α-protected amino acids having protected side chains are added stepwise to a growing polypeptide chain linked by its C-terminal and to an insoluble polymeric support i.e., polystyrene beads. The peptides are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art and so are not discussed in detail herein (See, Atherton, et al., 1989, Solid Phase Peptide Synthesis: A Practical Approach, IRL Press, and Bodanszky, 1993, Peptide Chemistry, A Practical Textbook, 2nd ed., Springer-Verlag).

Purification of the resulting HSP is accomplished using conventional procedures, such as preparative HPLC using gel permeation, partition and/or ion exchange chromatography. The choice of appropriate matrices and buffers are well known in the art and so are not described in detail herein.

### 4.7 Activated Antigen Presenting Cells (APCs)

In the various embodiments as above-described, in the place of an HSP preparation, activated APCs can be administered to a subject for a similar result. The invention includes a method of activating antigen presenting cells comprising contacting APCs with an HSP preparation. Prior to treatment with an HSP preparation to activate the APCs, the cells can optionally be enriched or purified, and/or expanded ex vivo by methods well known in the art. The APCs can be obtained from a subject, preferably the same subject to whom the treated APCs are re-administered (i.e., autologous APCs are used), although non-autologous APCs can also be used. The non-autologous APCs can be syngeneic (i.e., from an identical twin of the individual to which the activated APCs will be administered); or allogeneic (i.e., an individual who shares at least one common MHC allele with the individual to whom the activated APCs will be administered.) The activation of APCs can be monitored by techniques well known in the art, such as but not limited to those described in section 6 for testing CD11b⁺ cells. In a specific embodiment, the activated APCs can be used in vivo to produce or increase an immune response elicited by an immunoreactive reagent which is administered to the subject at reasonably the same time. The activated APCs can alternatively be administered within various time frames as discussed above, such as but not limited to a time frame of one to twenty four hours before or after the administration of an immunoreactive reagent, or periodically for a few days or more after a slow- or continuous-release type of immunoreactive reagent is used. Preferably, the treated APCs are administered to a site at or near the site of administration of the immunoreactive reagent. The administration of activated APCs can be conducted by any techniques known in the art.

### 4.8 Immunoreactive Reagents

Immunoreactive reagents include antibodies, molecules or proteins engineered to include the antigen binding portion of an antibody, molecules or proteins engineered to include an antigen binding domain that recognizes the target antigen of interest and a constant region domain that mediates antibody dependent immune responses, a peptide or domain that interacts specifically with the antigen of interest, or any antigen binding domain that interacts with an antigen/epitope of interest and the domain of the constant region of an antibody that mediates antibody dependent immune effector cell responses or processes. Examples of such domains or regions within the Ab constant region that can be used in the present invention include those disclosed in Reddy et al., 2000, J. Immunol. 164(4):1925-33; Coloma et al., 1997, Nat. Biotechnol. 15(2):159-63; Carayannopoulos et al., 1994, Proc Natl. Acad. Sci. U.S.A. 91(18):8348-52; Morrison, 1992, Annu. Recombinant Expression Vector Immunol. 10:239-65; Traunecker et al., 1992, Int. J. Cancer Suppl. 7:51-2; Gillies et al., 1990, Hum. Antibodies Hybridomas 1(1):47-54; each of which is incorporated herein by reference in its entirety.

Preferably, the immunoreactive reagents of the invention comprise 1) an antigen binding region and 2) a region that mediates one or more antibody dependent immunological processes. The antigen binding region can comprise or consist of the antigen binding region of an antibody. The antigen binding region can comprise any peptide or domain that interacts specifically with an antigen of interest. For example, the antigen binding region can be a ligand or other specific binding partner of the antigen of interest, or can be a fragment of such ligand or binding partner, or can be derived from such ligand or binding partner.

The region that mediates one or more antibody dependent immunological processes can comprise or consist of a region that is capable of binding an Fc receptor, e.g., the portion of an antibody that binds Fc receptors, or a region that binds complement, e.g., the complement binding region of an antibody. This region can also be an antigen binding domain of an antibody that binds to Fe receptors or complement.

Such antibody dependent processes include, but are not limited to, antibody dependent cellular cytotoxicity, activation of complement, opsonization and phagocytosis. The effector cells that mediate certain antibody dependent processes include monocytes, macrophages, natural killer cells, and polymorphonuclear cells. Without being bound by a particular mechanism, it is thought that HSPs are able to increase receptors on the effector cells responsible for mediating the antibody dependent response. These receptors include the Fc alpha and Fc gamma receptors, isoforms thereof, or any combination thereof. Thus, in a particular embodiment, the region of the immunoreactive reagent that mediates one or more antibody dependent immunological processes comprises or consists of a region that is a ligand for Fc receptors, preferably the Fc a receptor or the Fe gamma receptor, or both. In another embodiment, the region of the immunoreactive reagent that mediates one or more antibody dependent immunological processes comprises or consists of a region that stimulates the function of immune effector cells, preferably monocytes, macrophages, natural killer cells, polymorphonuclear cells, or any combination of two or more of such cells, such that a prophylactic and/or therapeutic effect is achieved. In certain embodiments, when the HSP preparation induces immune response such as cytokine release and/or T cell activation, the immunoreactive reagent potentiates or costimulates such immune responses.

In a preferred embodiment, the immunoreactive reagent is an antibody, or a composition comprising an antibody or antibodies such as serum. In a particular embodiment, the immunoreactive reagent is an IgA, IgG or IgM antibody, or comprises a fragment thereof. In a particularly preferred embodiment, the immunoreactive reagent is a monoclonal antibody, or includes fragments of a monoclonal antibody. The immunoreactive reagent may also comprise or consist of human immune globulin for treatment of Hepatitis B; Respigam for the treatment of RSV; Sandoglobulin, or ImmuneGlobulin IV (IGIV). In another embodiment, the immunoreactive reagent is not directed towards any single epitope, but instead comprises a mixture of one or more molecules that bind to a population of epitopes. An example of such an immunoreactive reagent is serum or antibodies concentrated from serum or plasma. Such serum or plasma may be from a subject immunized against a particular antigen, or from a subject not so immunized.

Antibodies that can be used in the methods of the invention include, but are not limited to, monoclonal antibodies, polyclonal antibodies, synthetic antibodies, multispecific antibodies, human antibodies, humanized antibodies, chimeric antibodies, single-chain Fvs (scFv), single chain antibodies, Fab fragments, F(ab) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies used in the methods of the present invention include immunoglobulin molecules and immunologically active portions of immunoglobulin molecules, i.e., molecules that contain an antigen binding site that immunospecifically binds to the target of interest. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

In another embodiment, the immunoreactive reagent is a bi-specific molecule having two antigen binding regions of different specificity, i.e., one recognizing an epitope on a target cell or protein, and the other recognizing an epitope of an effector cell, e.g., an epitope of FcR. In another embodiment, the immunoreactive reagent is a bi-specific molecule having two antigen binding domains for different epitopes on the target cell/protein, and a domain that mediates antibody dependent immune responses. Such bi-specific molecules that target cancer cells or pathogens and their therapeutic effects have been examined both in vivo and in vitro (e.g., Wallace et al., 2001, J Immunol. Methods 248(1-2):167-82; Sundarapandiyan et al., 2001, J. Immunol. Methods 248(1-2):113-23; Honeychurch et al., 2000, Blood 96(10):3544-52; Negri et al., 1995, Br J Cancer 72(4):928-33; Wang et al., 1994, Zhonghua Zhong Liu Za Zhi 16(2):83-7, Chinese) (each of which is incorporated herein by reference in its entirety).

In a preferred embodiment, the immunoreactive reagent is purified. “Purified” as used herein to describe certain peptides, antibodies, molecules, proteins, antigens, HSPs, HSP-peptide complexes, and the like, refer to a state beyond that in which the molecules, proteins, antigens, and the like, are separated from greater than 1%, 5%, 10%, 20%, 30%, 40%, 50%, 60%, 70%, 80%, 90%, 95%, or 99% of the proteins, polysaccharides, and/or lipids with which the peptides, antibodies, molecules, proteins, antigens, HSPs, HSP-peptide complexes, and the like are normally associated naturally. If the isolated molecules, proteins, antigens, HSPs, HSP-peptide complexes, and the like are synthesized, they are contaminated with less than 50%, 40%, 30%, 20%, 10%, 5%, 1% or 0.1% of the chemical precursors or synthesis reagents used to synthesize the molecules, proteins, antigens, HSPs, HSP-peptide complexes, and the like. In preferred embodiments the peptides, antibodies, molecules, proteins, antigens, HSPs, HSP-peptide complexes, and the like are at least 1% pure, 5% pure, 10% pure, 20% pure, 30% pure, 40% pure, 50% pure, 60% pure, 70% pure, 80% pure, 90% pure, 95% pure, 99% pure, or 100% pure. As used herein, the term “% pure” indicates the percentage of the total composition that is made up of the molecule of interest, by weight. Thus, a composition of 100 grams containing 50 grams of a molecule of interest is 50% pure with respect to the molecule of interest.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas, pp. 563-681 (Elsevier, N.Y., 1981) (both of which are incorporated herein by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

### 4.8.1 Preparation of Immunoreactive Reagents

The immunoreactive reagents of the invention can be produced by any method known in the art for the synthesis of antibodies, in particular, by chemical synthesis or preferably, by recombinant expression techniques. Such methods are described below with reference to an antibody immunoreactive reagent, but are readily applicable to the production of other immunoreactive reagents.

Methods for producing and screening for specific antibodies using hybridoma technology are routine and well known in the art. In a non-limiting example, mice can be immunized with an antigen of interest or a cell expressing such an antigen. Once an immune response is detected, e.g., antibodies specific for the antigen are detected in the mouse serum, the mouse spleen is harvested and splenocytes isolated. The splenocytes are then fused by well known techniques to any suitable myeloma cells. Hybridomas are selected and cloned by limiting dilution. The hybridoma clones are then assayed by methods known in the art for cells that secrete antibodies capable of binding the antigen. Ascites fluid, which generally contains high levels of antibodies, can be generated by inoculating mice intraperitoneally with positive hybridoma clones.

Antibody fragments which recognize specific epitopes may be generated by known techniques. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the complete light chain, and the variable region, the CH1 region and the hinge region of the heavy chain.

For example, antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains, such as Fab and Fv or disulfide-bond stabilized Fv, expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage, including fd and M13. The antigen binding domains are expressed as a recombinantly fused protein to either the phage gene III or gene VIII protein. Examples of phage display methods that can be used to make the immunoglobulins, or fragments thereof, of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired fragments, and expressed in any desired host, including mammalian cells, insect cells, plant cells, yeast, and bacteria, e.g., as described in detail below. For example, techniques to recombinantly produce Fab, Fab′ and F(ab)₂ fragments can also be employed using methods known in the art such as those disclosed in PCT publication WO 92/22324; Mullinax et al., 1992, BioTechniques 12(6):864-869; and Sawai et al., 1995, AJRI 34:26-34; and Better et al., 1988, Science 240:1041-1043 (each of which is incorporated by reference in its entirety). Examples of techniques which can be used to produce single-chain Fvs and antibodies include those described in U.S. Pat. Nos. 4,946,778 and 5,258,498; Huston et al., 1991, Methods in Enzymology 203:46-88; Shu et al., 1993, PNAS 90:7995-7999; and Skerra et al., 1988, Science 240:1038-1040.

For some uses, including in vivo use of antibodies in humans, it may be preferable to use chimeric, humanized, or human antibodies. A chimeric antibody is a molecule in which different portions of the antibody are derived from different animal species, such as antibodies having a variable region derived from a murine monoclonal antibody and a constant region derived from a human immunoglobulin. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; U.S. Pat. Nos. 5,807,715; 4,816,567; and 4,816,397, which are incorporated herein by reference in their entireties. Humanized antibodies are antibody molecules from non-human species that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Queen et al., U.S. Pat. No. 5,585,089; Reichmann et al., 1988, Nature 332:323, which are incorporated herein by reference in their entireties. Antibodies can be humanized using a variety of techniques known in the art including, for example, CDR-grafting (EP 239,400; PCT publication WO 91/09967; U.S. Pat. Nos. 5,225,539; 5,530,101 and 5,585,089), veneering or resurfacing (EP 592,106; EP 519,596; Padlan, 1991, Molecular Immunology 28(4/5):489-498; Studnicka et al., 1994, Protein Engineering 7(6):805-814; Roguska et al., 1994, Proc Natl. Acad. Sci. USA, 91:969-973), and chain shuffling (U.S. Pat. No. 5,565,332), all of which are hereby incorporated by reference in their entireties.

The long term use of therapeutic and/or prophylactic antibodies may be limited by the immunogenicity of the antibody which evokes host immune responses that limit their functional performance and use. Strategies, including the TolerMab™ technology (TolerRx, Cambridge, Mass.), have been developed to modify monoclonal antibodies in order to reduce antibody immunogenicity, thereby allowing for prolonged and/or recurrent administration of antibody therapeutics while avoiding neutralization by the host immune system. Accordingly, a “tolerized” monoclonal antibody which has been rendered capable of inducing tolerance to itself while maintaining the ability to target antigen and carry out its function in vivo may be desirable for the therapeutic or prophylactic treatment of patients in accordance with the present invention. Gilliland et al., 1999, J. Immunol. 162:3663-71.

Completely human antibodies are particularly desirable for therapeutic or prophylactic treatment of human patients. Human antibodies can be made by a variety of methods known in the art including phage display methods described above using antibody libraries derived from human immunoglobulin sequences. See U.S. Pat. Nos. 4,444,887 and 4,716,111; and PCT publications WO 98/46645; WO 98/50433; WO 98/24893; WO 98/16654; WO 96/34096; WO 96/33735; and WO 91/10741, each of which is incorporated herein by reference in its entirety.

Human antibodies can also be produced using transgenic mice which are incapable of expressing functional endogenous immunoglobulins, but which can express human immunoglobulin genes. For an overview of this technology for producing human antibodies, see Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entireties. In addition, companies such as Abgenix®, Inc. (Freemont, Calif.), Medarex® (NJ) and Genpharn® (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., 1988, Bio/technology 12:899-903).

In a preferred embodiment, the antibodies have in vivo therapeutic and/or prophylactic uses. Examples of therapeutic and prophylactic antibodies include, but are not limited to, MDX-010 (Medarex, N.J.) which is a humanized anti-CTLA-4 antibody currently in clinic for the treatment of prostate cancer; SYNAGIS® (MedImmune®, MD) which is a humanized anti-respiratory syncytial virus (RSV) monoclonal antibody for the treatment of patients with RSV infection; HERCEPTIN® (Trastuzumab) (Genentech®, CA) which is a humanized anti-HER2 monoclonal antibody for the treatment of patients with metastatic breast cancer; REMICADE® (infliximab) (Centocor®, PA) which is a chimeric anti-TNFα monoclonal antibody for the treatment of patients with Crone's disease; REOPRO® (abciximab) (Centocor®) which is an anti-glycoprotein IIb/IIIa receptor on the platelets for the prevention of clot formation; ZENAPAX® (daclizumab) (Roche Pharmaceuticals®, Switzerland) which is an immunosuppressive, humanized anti-CD25 monoclonal antibody for the prevention of acute renal allograft rejection. Other examples are a humanized anti-CD18 F(ab′)₂ (Genentech®); CDP860 which is a humanized anti-CD18 F(ab′)₂ (Celltech®, UK); PRO542 which is an anti-HIV gp120 antibody fused with CD4 (Progenics®/Genzyme Transgenics®); Ostavir which is a human anti Hepatitis B virus antibody (Protein Design Lab®/Novartis®); PROTOVIR™ which is a humanized anti-CMV IgG1 antibody (Protein Design Lab®/Novartis®); MAK-195 (SEGARD®) which is a murine anti-TNFα F(ab′)₂ (Knoll Pharma®/BASF®); IC14 which is an anti-CD14 antibody (ICOS Pharm®); a humanized anti-VEGF IgG1 antibody (Genentech®); OVAREX™ which is a murine anti-CA 125 antibody (Altarex®); PANOREX™ which is a murine anti-17-IA cell surface antigen IgG2a antibody (Glaxo Wellcome®/Centocor®); BEC2 which is a murine anti-idiotype (GD3 epitope) IgG antibody (ImClone System®); IMC-C225 which is a chimeric anti-EGFR IgG antibody (ImClone System®); VITAXIN™ which is a humanized antiαVβ3 integrin antibody (Applied Molecular Evolution®/MedImmune®); Campath 1H/LDP-03 which is a humanized anti CD52 IgG1 antibody (Leukosite®); Smart M195 which is a humanized anti-CD33 IgG antibody (Protein Design Lab®/Kanebo®); RITUXAN™ which is a chimeric anti-CD20 IgG1 antibody (IDEC Pharm®/Genentech®, Roche®/Zettyaku®); LYMPHOCIDE™ which is a humanized anti-CD22 IgG antibody (Immunomedics®); Smart ID10 which is a humanized anti-HLA antibody (Protein Design Lab®); ONCOLYM™ (Lym-1) is a radiolabelled murine anti-HLA DIAGNOSTIC REAGENT antibody (Techniclone®); ABX-IL8 is a human anti-IL8 antibody (Abgenix®); anti-CD11a is a humanized IgG1 antibody (Genentech®/Xoma®); ICM3 is a humanized anti-ICAM3 antibody (ICOS Pharm); IDEC-114 is a primatized anti-CD80 antibody (IDEC Pharm®/Mitsubishi®); ZEVALIN™ is a radiolabelled murine anti-CD20 antibody (IDEC®/Schering AG®); IDEC-131 is a humanized anti-CD40L antibody (IDEC®/Eisai®); IDEC-151 is a primatized anti-CD4 antibody (IDEC); IDEC-152 is a primatized anti-CD23 antibody (IDEC®/Seikagaku®); SMART anti-CD3 is a humanized anti-CD3 IgG (Protein Design Lab); 5G1.1 is a humanized anti-complement factor 5 (C5) antibody (Alexion Pharm®); D2E7 is a humanized anti-TNF-α antibody (CAT®/BASF®); CDP870 is a humanized anti-TNF-α Fab fragment (Celltech®); IDEC-151 is a primatized anti-CD4 IgG1 antibody (IDEC Pharm®/SmithKline Beecham®); MDX-CD4 is a human anti-CD4 IgG antibody (Medarex®/Eisai®/Genmab®); CDP571 is a humanized anti-TNF-α IgG4 antibody (Celltech®); LDP-02 is a humanized anti-α4β7 antibody (LeukoSite®/Genentech®); OrthoClone OKT4A is a humanized anti-CD4 IgG antibody (Ortho Biotech®); ANTOVA™ is a humanized anti-CD40L IgG antibody (Biogen®); ANTEGREN™ is a humanized anti-VLA-4 IgG antibody (Elan®); MDX-33 is a human anti-CD64 (FcγR) antibody (Medarex®/Centeon®); SCH55700 is a humanized anti-IL-5 IgG4 antibody (Celltech®/Schering®); SB-240563 and SB-240683 are humanized anti-IL-5 and IL-4 antibodies, respectively, (SmithKline Beecham®); rhuMab-E25 is a humanized anti-IgE IgG1 antibody (Genentech®/Norvartis®/Tanox Biosystems®); ABX-CBL is a murine anti CD-147 IgM antibody (Abgenix®); BTI-322 is a rat anti-CD2 IgG antibody (Medimmune®/Bio Transplant®); Orthoclone/OKT3 is a murine anti-CD3 IgG2a antibody (ortho Biotech®); SIMULECT™ is a chimeric anti-CD25 IgG1 antibody (Novartis Pharm®); LDP-01 is a humanized anti-β₂-integrin IgG antibody (LeukoSite); Anti-LFA-1 is a murine anti CD18 F(ab′)₂ (Pasteur-Merieux®/Immunotech®); CAT-152 is a human anti-TGF-β₂ antibody (Cambridge Ab Tech®); and Corsevin M is a chimeric anti-Factor VII antibody (Centocor®). The above-listed immunoreactive reagents, as well as any other immunoreactive reagents, may be administered according to any regimen known to those of skill in the art, including the regimens recommended by the suppliers of the immunoreactive reagents.

The nucleotide sequence encoding an antibody or other immunoreactive reagent may be obtained from any information available to those of skill in the art (i.e., from Genbank, the literature, or by routine cloning). If a clone containing a nucleic acid encoding a particular antibody or an epitope-binding fragment thereof or other immunoreactive reagent is not available, but the sequence of the antibody molecule or epitope-binding fragment thereof or other immunoreactive reagent is known, a nucleic acid encoding the immunoglobulin or other immunoreactive reagent may be chemically synthesized or obtained from a suitable source (e.g., an antibody cDNA library, or a cDNA library generated from, or nucleic acid, preferably poly A⁺ RNA, isolated from any tissue or cells expressing the antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art. In the case of immunoreactive reagents that do not exist in nature, nucleic acids encoding the different regions of the immunoreactive reagent can be obtained from preexisting libraries or known genes, or can be synthesized.

Once the nucleotide sequence of the antibody or other immunoreactive reagent is determined, the nucleotide sequence of the antibody or other immunoreactive reagent may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y.; and Ausubel et al., eds., 1998, Current Protocols in Molecular Biology, John Wiley & Sons, NY, which are both incorporated by reference herein in their entireties), to generate antibodies or other immunoreactive reagent having a different amino acid sequence by, for example, introducing amino acid substitutions, deletions, and/or insertions into the epitope-binding domain regions of the antibodies or other immunoreactive reagent or into the constant (Fc) regions of the antibodies or other immunoreactive reagent which are involved in the interaction with immune effector cells.

Recombinant expression of an antibody or other immunoreactive reagent requires construction of an expression vector containing a nucleotide sequence that encodes the antibody or other immunoreactive reagent. Once a nucleotide sequence encoding an antibody molecule or a heavy or light chain of an antibody, or portion thereof (preferably, but not necessarily, containing the heavy or light chain variable region) or other immunoreactive reagent has been obtained, the vector for the production of the antibody molecule or other immunoreactive reagent may be produced by recombinant DNA technology using techniques well known in the art. Thus, methods for preparing a protein by expressing a polynucleotide containing an antibody or other immunoreactive reagent encoding nucleotide sequence are described herein. Methods which are well known to those skilled in the art can be used to construct expression vectors containing antibody or other immunoreactive reagent coding sequences and appropriate transcriptional and translational control signals. These methods include, for example, in vitro recombinant DNA techniques, synthetic techniques, and in vivo genetic recombination. The nucleotide sequence encoding the heavy-chain variable or constant region, light-chain variable or constant region, both the heavy-chain and light-chain variable regions, an epitope-binding fragment of the heavy- and/or light-chain variable region, or one or more complementarity determining regions (CDRs) of an antibody or other immunoreactive reagent may be cloned into such a vector for expression. The expression vector is transferred to a host cell by conventional techniques and the transfected cells are then cultured by conventional techniques.

A variety of host-expression vector systems may be utilized to express the antibody molecules or other immunoreactive reagent of the invention. Such host-expression systems represent vehicles by which the coding sequences of interest may be produced and subsequently purified, but also represent cells which may, when transformed or transfected with the appropriate nucleotide coding sequences, express an antibody molecule or other immunoreactive reagent of the invention in situ. These include, but are not limited to, microorganisms such as bacteria (e.g., E. coli and B. subtilis) transformed with recombinant bacteriophage DNA, plasmid DNA or cosmid DNA expression vectors containing antibody or other immunoreactive reagent coding sequences; yeast (e.g., Saccharomyces and Pichia) transformed with recombinant yeast expression vectors containing antibody or other immunoreactive reagent coding sequences; insect cell systems infected with recombinant virus expression vectors (e.g., baculovirus) containing antibody or other immunoreactive reagent coding sequences; plant cell systems infected with recombinant virus expression vectors (e.g., cauliflower mosaic virus, CaMV; and tobacco mosaic virus, TMV) or transformed with recombinant plasmid expression vectors (e.g., Ti plasmid) containing antibody or other immunoreactive reagent coding sequences; and mammalian cell systems (e.g., COS, CHO, BHK, 293, 3T3 and NSO cells) harboring recombinant expression constructs containing promoters derived from the genome of mammalian cells (e.g., metallothionein promoter) or from mammalian viruses (e.g., the adenovirus late promoter, the vaccinia virus 7.5K promoter). Preferably, bacterial cells such as Escherichia coli, and more preferably, eukaryotic cells, especially for the expression of whole recombinant antibody molecule or other immunoreactive reagent, are used for the expression of a recombinant antibody or other immunoreactive reagent molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101, and Cockett et al., 1990, Bio/Technology 8:2).

In bacterial systems, a number of expression vectors may be advantageously selected depending upon the use intended for the antibody molecule or other immunoreactive reagent being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody or other immunoreactive reagent coding sequence may be ligated individually into the vector in frame with the lacZ coding region so that a fusion protein is produced; and pIN vectors (Inouye & Inouye, 1985, Nucleic Acids Res. 13:3101-3109, and Van Heeke & Schuster, 1989, J. Biol. Chem. 24:5503-5509).

In an insect system, Autographa californica nuclear polyhedrosis virus (AcNPV) is used as a vector to express foreign genes. The virus grows in Spodoptera frugiperda cells. The antibody or other immunoreactive reagent coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized to express an antibody molecule or other immunoreactive reagent of the invention. In cases where an adenovirus is used as an expression vector, the antibody or other immunoreactive reagent coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule or other immunoreactive reagent in infected hosts (e.g., see Logan & Shenk, 1984, Proc. Natl. Acad. Sci. USA 81:355-359). Specific initiation signals may also be required for efficient translation of inserted antibody or other immunoreactive reagent coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bitter et al., 1987, Methods in Enzymol. 153:516-544).

In addition, a host cell strain may be chosen which modulates the expression of the antibody or other immunoreactive reagent sequences, or modifies and processes the antibody or other immunoreactive reagent in the specific fashion desired. Such modifications (e.g., glycosylation) and processing (e.g., cleavage) of protein products may be important for the function of the antibody or other immunoreactive reagent. Different host cells have characteristic and specific mechanisms for the post-translational processing and modification of proteins and gene products. Appropriate cell lines or host systems can be chosen to ensure the correct modification and processing of the antibody or other immunoreactive reagent expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, W138, and in particular, myeloma cells such as NSO cells, and related cell lines, see, for example, Morrison et al., U.S. Pat. No. 5,807,715, which is hereby incorporated by reference in its entirety.

For long-term, high-yield production of recombinant antibodies or other immunoreactive reagent, stable expression is preferred. For example, cell lines which stably express the antibody molecule or other immunoreactive reagents may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule or other immunoreactive reagent. Such engineered cell lines may be particularly useful in screening and evaluation of compositions that interact directly or indirectly with the antibody molecule or other immunoreactive reagent.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk⁻, hgprt⁻ or aprt⁻ cells, respectively. Also, antimetabolite resistance can be used as the basis of selection for the following genes: dhfr, which confers resistance to methotrexate (Wigler et al., 1980, Natl. Acad. Sci. USA 77:357, and O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; and May, 1993, TIB TECH 11(5):155-215); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), 1993, Current Protocols in Molecular Biology, John Wiley & Sons, NY; Kriegler, 1990, Gene Transfer and Expression, A Laboratory Manual, Stockton Press, NY; in Chapters 12 and 13, Dracopoli et al. (eds), 1994, Current Protocols in Human Genetics, John Wiley & Sons, NY; and Colberre-Garapin et al., 1981, J. Mol. Biol. 150:1, which are incorporated by reference herein in their entireties.

The expression levels of an antibody molecule or other immunoreactive reagent can be increased by vector amplification (for a review, see Bebbington and Hentschel, 1987, The use of vectors based on gene amplification for the expression of cloned genes in mammalian cells in DNA cloning, Vol. 3. Academic Press, New York). When a marker in the vector system expressing antibody or other immunoreactive reagent is amplifiable, increase in the level of inhibitor present in culture of host cell will increase the number of copies of the marker gene. Since the amplified region is associated with the antibody or other immunoreactive reagent gene, production of the antibody or other immunoreactive reagent will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides or different selectable markers to ensure maintenance of both plasmids. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody or other immunoreactive reagent molecule of the invention has been produced by recombinant expression, it may be purified by any method known in the art for purification of an immunoglobulin molecule or other immunoreactive reagent, for example, by chromatography (e.g., ion exchange, affinity, particularly by affinity for the specific antigen after Protein A purification, and sizing column chromatography), centrifugation, differential solubility, or by any other standard techniques for the purification of proteins. Further, the antibodies or other immunoreactive reagents of the present invention or fragments thereof may be fused to heterologous polypeptide sequences described herein or otherwise known in the art to facilitate purification.

The present invention also encompasses the use of antibodies or fragments thereof recombinantly fused or chemically conjugated (including both covalent and non-covalent conjugations) to a heterologous polypeptide (or portion thereof, preferably to a polypepetide of at least 10, at least 20, at least 30, at least 40, at least 50, at least 60, at least 70, at least 80, at least 90 or at least 100 amino acids) to generate fusion proteins. The fusion does not necessarily need to be direct, but may occur through linker sequences. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., PCT publication WO 93/21232; EP 439,095; Naramura et al., Immunol. Lett. 39:91-99 (1994); U.S. Pat. No. 5,474,981; Gillies et al., PNAS 89:1428-1432 (1992); and Fell et al., J. Immunol. 146:2446-2452(1991), which are incorporated by reference in their entireties.

The present invention further includes compositions comprising heterologous polypeptides fused or conjugated to antibody fragments. For example, the heterologous polypeptides may be fused or conjugated to a Fab fragment, Fc fragment, Fv fragment, F(ab)₂ fragment, or portion thereof. Methods for fusing or conjugating polypeptides to antibody portions are known in the art. See, e.g., U.S. Pat. Nos. 5,336,603, 5,622,929, 5,359,046, 5,349,053, 5,447,851, and 5,112,946; EP 307,434; EP 367,166; PCT publication Nos. WO 96/04388 and WO 91/06570; Ashkenazi et al., Proc. Natl. Acad. Sci. USA 88: 10535-10539 (1991); Zheng et al., J. Immunol. 154:5590-5600 (1995); and Vil et al., Proc. Natl. Acad. Sci. USA 89:11337-11341(1992) (said references incorporated by reference in their entireties).

The present invention further encompasses uses of antibodies or fragments thereof conjugated to a therapeutic agent.

An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, grarnicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g. vincristine and vinblastine).

Further, an antibody or fragment thereof may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567-1574), and VEGI (see, International Publication No. WO 99/23105), a thrombotic agent or an anti-angiogenic agent, e.g., angiostatin or endostatin; or, a biological response modifier such as, for example, a lympholine (e.g., interleulin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Moreover, an antibody can be conjugated to therapeutic moieties such as a radioactive metal ion, such as alph-emiters such as ²¹³Bi or macrocyclic chelators useful for conjugating radiometal ions, including but not limited to, ¹³¹In, ¹³¹LU, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50 each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody can be conjugated to a second antibody to form an antibody heteroconjugate as described by Segal in U.S. Pat. No. 4,676,980, which is incorporated herein by reference in its entirety.

### 4.9 Therapeutic/Prophylactic Utility

### Determination of Immunogenicity of Immunoreactive Reagents After HSP Treatment

In an optional procedure, the production of or increase in immunogenicity of an immunoreactive reagent that is used with the HSP preparation of the invention can be assessed using various methods well known in the art and exemplified in Section 5.

In other methods, the “tetramer staining” assay (Altman et al., 1996, Science 274: 94-96) may be used to identify antigen-specific T-cells. For example, in one embodiment, an MHC molecule containing a specific peptide antigen, such as a tumor-specific antigen, is multimerized to make soluble peptide tetramers and labeled, for example, by complexing to streptavidin. The MHC-peptide antigen complex is then mixed with a population of T cells obtained from a patient treated with an immunoreactive reagent and the HSP preparation. Biotin is then used to stain T cells which express the tumor-specific antigen of interest.

Furthermore, using the mixed lymphocyte target culture assay, the cytotoxicity of T cells can be tested in a 4 hour ⁵¹Cr-release assay (see Palladino et al., 1987, Cancer Res. 47:5074-5079). In this assay, the mixed lymphocyte culture is added to a target cell suspension to give different effector:target (E:T) ratios (usually 1:1 to 40:1). The target cells are pre-labeled by incubating 1×10⁶ target cells in culture medium containing 500 μCi of ⁵¹Cr per ml for one hour at 37° C. The cells are washed three times following labeling. Each assay point (E:T ratio) is performed in triplicate and the appropriate controls incorporated to measure spontaneous ⁵¹Cr release (no lymphocytes added to assay) and 100% release (cells lysed with detergent). After incubating the cell mixtures for 4 hours, the cells are pelleted by centrifugation at 200 g for 5 minutes. The amount of ⁵¹Cr released into the supernatant is measured by a gamma counter. The percent cytotoxicity is measured as cpm in the test sample minus spontaneously released cpm divided by the total detergent released cpm minus spontaneously released cpm. In order to block the MHC class I cascade a concentrated hybridoma supernatant derived from K-44 hybridoma cells (an anti-MHC class I hybridoma) is added to the test samples to a final concentration of 12.5%.

Alternatively, the ELISPOT assay can be used to measure cytokine release in vitro by cytotoxic T cells after stimulation with an immunoreactive reagent and an HSP preparation. Cytokine release is detected by antibodies which are specific for a particular cytokine, such as interleukin-2, tumor necrosis factor α or interferon-γ (for example, see Scheibenbogen et al., 1997, Int. J. Cancer 71:932-936). The assay is carried out in a microtitre plate which has been pre-coated with an antibody specific for a cytokine of interest which captures the cytokine secreted by T cells. After incubation of T cells for 24-48 hours in the coated wells, the cytotoxic T cells are removed and replaced with a second labeled antibody that recognizes a different epitope on the cytokine. After extensive washing to remove unbound antibody, an enzyme substrate which produces a colored reaction product is added to the plate. The number of cytokine-producing cells is counted under a microscope. This method has the advantages of short assay time, and sensitivity without the need of a large number of cytotoxic T cells.

### 4.10 Pharmaceutical Compositions

The present invention also provides pharmaceutical compositions. Such prophylactically or therapeutically effective compositions comprise an immunoreactive reagent and an HSP, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of the immunoreactive reagent and HSP, preferably in purified form, together with a suitable amount of carrier so as to provide the form for proper administration to the patient. The formulation should suit the mode of administration.

The immunoreactive reagents and HSPs of this invention may also be advantageously utilized in combination with one or more drugs used to treat a disease, disorder, or infection such as, for example anti-cancer agents, anti-inflammatory agents, or anti-bacterial/fungal or anti-viral agents. Examples of anti-cancer agents include, but are not limited to cisplatin, carboplatin, cyclophosphamide, doxorubicin, etoposide, ifosfamide, paclitaxel, taxanes, CPT-11, topotecan, gemcitabine, oncovin, vinorelbine, oxaliplatin, 5-fluorouracil (5-FU), leucovorin, levamisole, BCNU, vinorelbine, temodar, vincristine and taxol.

Various delivery systems are known and can be used to administer the therapeutic and prophylactic agents encompassed by the invention, i.e. immunoreactive reagents and HSPs, e.g., encapsulation in liposomes, microparticles, microcapsules, recombinant cells capable of expressing the immunoreactive reagent, HSP preparation, the antibody or antibody fragment, receptor-mediated endocytosis (see, e.g., Wu and Wu, 1997, J. Biol. Chem. 262:4429-4432), construction of a nucleic acid as part of a retroviral or other vector, etc. Methods of administering an immunoreactive reagent or HSP preparation or pharmaceutical compositions comprising the same include, but are not limited to, parenteral administration (e.g., intradermal, intramuscular, intraperitoneal, intravenous and subcutaneous), epidural, and mucosal (e.g., intranasal and oral routes). In a specific embodiment, immunoreactive reagents, for example, antibodies, are administered intramuscularly, intravenously, or subcutaneously. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985, 320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety. In one embodiment, a therapeutic or prophylactic agent is administered using Alkermes AIR™ pulmonary drug delivery technology (Alkermes, Inc., Cambridge, Mass.).

Solubility and the site of the administration are factors which should be considered when choosing the route of administration. The mode of administration can be varied, including, but not limited to, e.g., subcutaneously, intravenously, intraperitoneally, intramuscularly, intradermally or mucosally. Mucosal routes can further take the form of oral, rectal and nasal administration. With the above factors taken into account, it is preferable to administer a first therapeutic or prophylactic agent to a site that is the same or proximal to the site of administration of the second agent. In a method for treating a tumor, the HSP preparation is administered in proximity to the tumor, most preferably by intratumoral injection.

In an embodiment of the invention, HSP preparations and immunoreactive reagents may be administered using any desired route of administration. Advantages of intradermal administration include use of lower doses and rapid absorption, respectively. Advantages of subcutaneous or intramuscular administration include suitability for some insoluble suspensions and oily suspensions, respectively. Mucosal routes of administration include, but are not limited to, oral, rectal and nasal administration. Preparations for mucosal administrations are suitable in various formulations as described below.

In another embodiment, the therapeutic or prophylactic agents of the invention are administered intramuscularly, intravenously, or subcutaneously. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents.

In a specific embodiment, it may be desirable to administer the pharmaceutical compositions of the invention locally to the area in need of treatment or prevention. In one embodiment, the treatment or prevention may be achieved by, for example, and not by way of limitation, local infusion, by injection, or by means of an implant, said implant being of a porous, non-porous, or gelatinous material, including membranes, such as sialastic membranes, or fibers. Preferably, care is taken to use materials to which the agent does not absorb.

In another embodiment, the composition can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the composition can be delivered in a controlled release or sustained release system. Any technique known to one of skill in the art can be used to produce sustained release formulations comprising one or more antibodies, or one or more fusion proteins. See, e.g., U.S. Pat. No. 4,526,938; PCT publication WO 91/05548; PCT publication WO 96/20698; Ning et al., 1996, “Intratumoral Radioimmunotheraphy of a Human Colon Cancer Xenograft Using a Sustained-Release Gel,” Radiotherapy & Oncology 39:179-189; Song et al., 1995, “Antibody Mediated Lung Targeting of Long-Circulating Emulsions,” PDA Journal of Pharmaceutical Science & Technology 50:372-397; Cleek et al., 1997, “Biodegradable Polymeric Carriers for a bFGF Antibody for Cardiovascular Application,” Pro. Intl. Symp. Control. Rel. Bioact. Mater. 24:853-854; and Lam et al., “Microencapsulation of Recombinant Humanized Monoclonal Antibody for Local Delivery,” Proc. Int'l. Symp. Control Rel. Bioact. Mater. 24:759-760, 1997, each of which is incorporated herein by reference in its entirety. In one embodiment, a pump may be used in a controlled release system (see Langer, supra; Sefton, 1987, CRC Crit. Ref. Biomed. Eng. 14:20; Buchwald et al., 1980, Surgery 88:507; and Saudek et al., 1989, N. Engl. J. Med. 321:574). In another embodiment, polymeric materials can be used to achieve controlled release of immunoreactive reagents or HSP preparations (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253). In yet another embodiment, a controlled release system can be placed in proximity of the therapeutic target (e.g., the lungs), thus requiring only a fraction of the systemic dose (see, e.g., Goodson, in Medical Applications of Controlled Release, supra, vol. 2, pp. 115-138 (1984)).

Other controlled release systems are discussed in the review by Langer, 1990, Science 249:1527-1533.

In one preferred embodiment, the HSP preparation is administered concurrently with the administration of an immunoreactive reagent. Concurrent administration of an HSP preparation and an immunoreactive reagent means that the HSP or HSP-peptide complex is co-administered as a mixture with, or administered separately from, but at reasonably the same time as the immunoreactive reagent. This method provides that the two administrations are performed within a time frame of less than one minute to about five minutes, or up to about sixty minutes from each other, for example, at the same doctor's visit.

In a preferred embodiment, the invention provides for a method of introducing an HSP preparation including, but not limited to, hsp60, hsp70, hsp90, hsp110, gp96, grp170, or calreticulin, alone or in combination with each other into a subject concurrently with the administration of an immunoreactive reagent at the same site or at a site in close proximity.

If the contemplated therapeutic or prophylactic agent is water-soluble, then it may be formulated in an appropriate buffer, for example, phosphate buffered saline or other physiologically compatible solutions, preferably sterile. Alternatively, if the resulting complex has poor solubility in aqueous solvents, then it may be formulated with a non-ionic surfactant such as Tween, or polyethylene glycol. Thus, the compounds and their physiologically acceptable solvates may be formulated for administration by inhalation or insufflation (either through the mouth or the nose) or oral, buccal, parenteral, or rectal administration or, in the case of tumors, directly injected into a solid tumor.

For oral administration, the pharmaceutical preparation may be in liquid form, for example, solutions, syrups or suspensions, or may be presented as a drug product for reconstitution with water or other suitable vehicle before use. Such a liquid preparation may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g. almond oil, oily esters, or fractionated vegetable oils); and preservatives (e.g., methyl or propyl-p-hydroxybenzoates or sorbic acid). The pharmaceutical preparation may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

The composition for oral administration may be suitably formulated to give controlled release of the active compound.

For buccal administration, the composition may take the form of tablets or lozenges formulated in conventional manner.

The agents may be formulated for parenteral administration by injection, e.g., by bolus injection or continuous infusion. Formulations for injection may be presented in unit dosage form, e.g., in ampoules or in multi-dose containers, with an added preservative. The preparation may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions may also be formulated in a rectal preparation such as a suppository or retention enema, e.g., containing conventional suppository bases such as cocoa butter or other glycerides.

In addition to the formulations described previously, the agents of the invention may also be formulated as a depot preparation. Such long acting formulations may be administered by implantation (for example, subcutaneously or intramuscularly) or by intramuscular injection. Thus, for example, the preparation may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

For administration by inhalation, the preparation for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Formulations of pharmaceutical compositions comprising HSPs and procedures for their manufacture can be found in the literature and in the U.S. patents incorporated by reference into this document.

The invention also provides that an immunoreactive reagent, for example an antibody, or an HSP preparation is packaged in a hermetically sealed container such as an ampoule or sachette indicating the quantity of immunoreactive reagent In one embodiment, the immunoreactive reagent and HSP are supplied together or separately as dry sterilized lyophilized powders or water free concentrates in one or more hermetically sealed containers and can be reconstituted, e.g., with water or saline to the appropriate concentration for administration to a subject. The effective dosage of each immunoreactive reagent can be estimated initially from in vitro assays. It also depends on the nature of the target antigen, the density of the antigen in the tumors, the tumor type, the manner of administration, which can be optimized by a person skilled in the art without undue experimentation. Usual effective dosages for injection range from about 0.1 to 5 mg/kg/day, preferably from about 1 to 4 mg/kg/day, and more preferably from 2 to 4 mg/kg/week. Preferably, the immunoreactive reagent is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, or at least 75 mg.

In a specific embodiment, immunoreactive reagents administered to an animal are of a species origin or species reactivity that is the same species as that of the animal. Thus, in a preferred embodiment, human or humanized antibodies are administered to a human patient for therapy or prophylaxis.

Depending on the route of administration and the type of HSPs in the HSP preparation, the amount of HSP in the HSP preparation can range, for example, from 0.1 to 1000 μg per administration. The preferred amounts of gp96 or hsp70 are in the range of 10 to 600 μg per administration and 0.1 to 50 μg, preferably 10 to 25 μg, if the HSP preparation is administered intradermally. For hsp 90, the preferred amounts are about 50 to 1000 μg per administration, and about 5 to 50 μg for intradermal administration.

In other embodiments, the heat shock protein is hsp60, hsp70, hsp90, gp96, or calreticulin. The dosage of HSP preparation to be administered depends to a large extent on the condition and size of the subject being treated as well as the amount of immunoreactive reagent administered, the frequency of treatment and the route of administration. Regimens for continuing therapy, including site, dose and frequency may be guided by the initial response and clinical judgment.

The optimal amount of a specific HSP for use with a specific composition of the invention may vary. Optimization of the specific HSP amount for a given composition is, as demonstrated by the examples cited above, well within the purview of the skilled artisan.

Because of the administration of the HSP preparation, a lesser amount of immunoreactive reagent may be required to elicit an immune response in a subject. The amount of immunoreactive reagent to be used with an HSP preparation, including amounts in the sub-optimal range, can be determined by dose-response experiments conducted in animal models by methods well known in the art.

In a preferred embodiment, the heat shock protein is hsp70. The amount of hsp70 in the pharmaceutical compositions is preferably in the range of 10 to 600 μg per administration and 0.1 to 50 μg, preferably 10 to 25 μg if the HSP preparation is administered intradermally.

In a particularly preferred embodiment, the heat shock protein is gp96. The amount of gp96 in the pharmaceutical compositions is preferably in the range of 10 to 600 μg per administration and 0.1 to 50 μg, preferably 10 to 25 μg if the HSP preparation is administered intradermally.

In an alternative embodiment, an immunoreactive reagent and HSP are supplied in liquid form in a hermetically sealed container indicating the quantity and concentration of the HSP and immunoreactive reagent. Preferably, the liquid form of the immunoreactive reagent is supplied in a hermetically sealed container at least 1 mg/ml, more preferably at least 2.5 mg/ml, at least 5 mg/ml, at least 8 mg/ml, at least 10 mg/ml, at least 15 mg/ml, or at least 25 mg/ml. Preferably, the liquid form of the HSP is supplied in a hermetically sealed container at least 0.1 mg/ml, more preferably at least 1.0 mg/ml, at least 5 mg/ml, at least 10 mg/ml, at least 25 mg/ml, at least 50 mg/ml, at least 100 mg/ml, or at least 250 mg/ml.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

Generally, the ingredients of compositions of the invention are supplied as a kit either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. In another embodiment, a kit of the invention further comprises a needle or syringe, preferably packaged in sterile form, for injecting the composition, and/or a packaged alcohol pad. Instructions are optionally included for administration of the compositions of the invention by a clinician or by the patient.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the composition of the invention which will be effective in the treatment, prevention or amelioration of one or more symptoms associated with a disease, disorder, or infection can be determined by standard clinical techniques. The precise dose to be employed in the formulation will depend on the route of administration, the age of the subject, and the seriousness of the disease, disorder, or infection, and should be decided according to the judgment of the practitioner and each patient's circumstances. Effective doses may be extrapolated from dose-response curves derived from in vitro or animal model (e.g., the cotton rat or Cynomolgous monkey) test systems. Models and methods for evaluation of the effects of HSPs and antibodies, or other immunoreactive reagents are known in the art. (Wooldridge et al., 1997, Blood 89(8): 2994-2998, incorporated by reference herein in its entirety).

For antibodies, the therapeutically or prophylactically effective dosage administered to a subject is typically 0.1 mg/kg to 200 mg/kg of the subject's body weight. Preferably, the dosage administered to a subject is between 0.1 mg/kg and 20 mg/kg of the subject's body weight and more preferably the dosage administered to a subject is between 1 mg/kg to 10 mg/kg of the subject's body weight. The dosage will, however, depend upon the extent to which the serum half-life of the molecule has been increased. Generally, human antibodies have longer half-lives within the human body than antibodies from other species due to the immune response to the foreign polypeptides. Thus, lower dosages of human antibodies and less frequent administration is often possible. Further, the dosage and frequency of administration of immunoreactive reagents may be reduced also by enhancing uptake and tissue penetration (e.g., into the lung) of the immunoreactive reagents such as, for example, lipidation. Specific antibody dosage information may be also be found in the manufacturer's insert for said antibody, or the Physician's Desk Reference (56^{th} ed., 2002).

Treatment of a subject with a therapeutically or prophylactically effective amount of an immunoreactive reagent and HSP can include a single treatment or, preferably, can include a series of treatments. In a preferred example, a subject is treated with an immunoreactive reagent in the range of between about 0.1 to 30 mg/kg body weight, one time per week for between about 1 to 10 weeks, preferably between 2 to 8 weeks, more preferably between about 3 to 7 weeks, and even more preferably for about 4, 5, or 6 weeks. Immunoreactive reagents and their dosages, routes of administration and recommended usage are known in the art and have been described in such literature as the Physician's Desk Reference (56^{th} ed., 2002) In a preferred example, a subject is treated with an HSP in the range of between about 0.1 to 1000 mg, more preferably, 1 to 500 mg, most preferably 2 to 250 mg, one time per week for between about 1 to 10 weeks, preferably between 2 to 8 weeks, more preferably between about 3 to 7 weeks, and even more preferably for 4, 5, or 6 weeks. One skilled in the art would be able to envision the appropriate HSP dosage depending on the condition to be treated and the immunoreactive reagent administered, as well as the subject.

In certain embodiments of the invention, compositions of the invention comprises HSPs in combination with excipients. Preferably, the heat shock protein is hsp60, hsp70, hsp90, gp96, or calreticulin, and the excipients are selected from nonionic surfactants, polyvinyl pyrolidone, human serum albumin, and various unmodified and derivatized cyclodextrins. More preferably, in these embodiments, the nonionic surfactants are selected from Polysorbate 20, Polysorbate-40, Polysorbate-60, and Polysorbate-80. The polyvinyl pyrolidone may preferably be Plasdone C15, a pharmaceutical grade of polyvinyl pyrolidone. Preferred cyclodextrins are hydroxypropyl-β-cyclodextrin, hydroxypropyl-γ-cyclodextrin, and methyl-β-cyclodextrin. Preferably, the cyclodextrins are β-cyclodextrins. Preferably, compositions of the invention comprise a prophylactically or therapeutically effective amount of HSP preparation or immunoreactive reagent, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The preparation may, if desired, be presented in a pack or dispenser device which may contain one or more unit dosage forms containing the HSP preparation. The pack may for example comprise metal or plastic foil, such as a blister pack. The pack or dispenser device may be accompanied by instructions for administration.

Compositions of the present invention can be administered to an animal, preferably a mammal and most preferably a human, to treat, prevent or ameliorate one or more symptoms associated with a disease, disorder, or infection. In a preferred embodiment, the composition of the invention exists outside of the body. Preferably, the immunoreactive reagent of the invention has been established to have some therapeutic benefit in the absence of heat shock protein, and recognizes an epitope on a cell or molecule associated with the cause or symptoms of a disease, disorder or infection.

The compositions comprise an immunoreactive reagent (i.e., an antigen binding protein comprising an antigen binding region and a region that mediates one or more antibody dependent immunological processes, e.g., an Fc receptor-binding region) and an HSP.

Each composition of the invention should contain at least one immunoreactive reagent (as defined herein, e.g., an antibody) and an HSP, and the compositions of the invention can also be used in conjunction with other forms of therapy for a particular disease. One or more immunoreactive reagents that immunospecifically bind to one or more target antigens may be used locally or systemically in the body as a prophylactic or a therapeutic agent.

### 4.11 Kits

Kits are also provided for carrying out the methods of the present invention, In a specific embodiment, a kit comprises a first container containing a heat shock protein preparation in an amount effective to increase an immune response elicited by an immunoreactive reagent against a target of the immunoreactive reagent against which an immune response is desired; and a second container containing the immunoreactive reagent in an amount that, when administered before, concurrently with, or after the administration of the heat shock protein preparation in the first container, is effective to induce an immune response against the target.

Kits of the invention are provided that comprise in a container an immunoreactive reagent in an amount effective to treat or prevent a disease or disorder; and in another container a heat shock protein preparation in an amount effective to increase or boost an immune response elicited by the immunoreactive reagent. In an embodiment, the amount of immunoreactive reagent present in the container is sub-optimal for inducing an immune response in a subject if administered independent of the heat shock protein preparation in the other container. The kit may optionally be accompanied by instructions.

The invention also provides kits comprising one or more containers with one or more of the ingredients of the pharmaceutical compositions of the invention. Optionally associated with such kit(s) can be a notice in the form prescribed by a governmental agency regulating the manufacture, use or sale of pharmaceuticals or biological products, which notice reflects approval by the agency of manufacture, use or sale for human administration. In one embodiment, the kits can optionally further comprise a predetermined amount of the immunoreative reagent (i.e., an antigen binding protein comprising an antigen binding region and a region that mediates one or more antibody dependent immunological processes, e.g., an Fc receptor-binding region) and an HSP. In a preferred embodiment, the kit comprises the immunoreactive reagent and the HSP in separate containers.

### 5. EXAMPLES

### 5.1 Enhancement of Antibody Mediated Lysis in vitro

Murine splenocytes (effector cells) are generated from the spleens of naive 6-8 week old mice. These effector cells are incubated with an appropriate amount of an HSP preparation and an appropriate amount of a monoclonal antibody for 24 to 72 hours. At the end of the incubation period, target cells (E.G7-OVA or MO4) are loaded with 51Cr. Effector cells and labeled target cells are incubated at defined effector:target ratios in the presence and absence of antibody to SIINFEKL/Class I MHC (1 to 10 ug/ml) at 4° C. for 30 to 60 minutes. The lysis in the presence of HSP and antibody is compared to controls without HSP, without antibody, or both.

### 5.2 Improvement of Protection in Tumor Challenge Model

C57B1/6 mice are inoculated by SC route in the flank with MO4 tumor (1×10⁵) or EG7-OVA tumor. At 24 to 48 hours after inoculation, mice are injected by IP route with an appropriate amount of an immunoreactive reagent (i.e., antibody) to SIINFEKL/Class I MHC, or by local SC route in the presence or absence of a appropriate amount of an HSP preparation. The antitumor effect of the combination treatment is compared to that of antibody treatment alone by monitoring the growth of the tumors over a 30 to 60 day period (measurement with calipers). Survival is determined, and significance with respect to time to death are assessed using Cox regression analysis. Mice are also observed daily for signs of toxicity including level of activity, ruffled fur, diarrhea, and general appearance. Models and methods for evaluation of the effects of antibodies, or other immunoreactive reagents are known in the art. (Wooldridge et al., 1997, Blood 89(8): 2994-2998, incorporated by reference herein in its entirety).

### 5.3 Improved Opsonization of Bacteria

Improved opsonization of bacteria by addition of an HSP preparation to therapeutic antibody treatment is demonstrated in vitro by incubating effector cells for the opsonophagocytosis assay (HL-60) with HSP preparation. The cells are evaluated for whether they are more effective in opsonizing S. pneumonia or S. aureus at a given antibody titer (for example a human serum sample with opsonizing activity specific for S. pneumonia or S. aureus, respectively).

### 5.4 Upregulation of Fc Receptors

Monocytes, natural killer cells, or polymorphonuclear cells are incubated in the presence or absence of an appropriate amount of an HSP preparation. The trypsinized cells are incubated at 4° C. for 60 min with monoclonal antibodies specific to Fc αR, Fc gamma R1, Fc gamma RII, or Fc gamma RIII. The cells are then incubated with an anti-mouse IgG FITC probe, washed, fixed in paraformaldehyde, and analyzed by FACScan. Upregulation of Fc receptors on these cells is monitored.

In addition, upregulation of TNF-alpha, IL-6 and MIP-1-alpha by HSP preparation in macrophage cells are also monitored.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A method of immunotherapy comprising administering to a subject in need thereof a purified HSP preparation and a purified immunoreactive reagent.

2. A method for improving the outcome of a passive immunization treatment in a subject receiving an immunoreactive reagent comprising administering a purified HSP preparation to said subject.

3. A method of treating cancer in a subject in need thereof comprising administering to said subject a purified HSP preparation and a purified immunoreactive reagent that is a cancer therapeutic.

4-6. (canceled)

7. A method of enhancing or inducing an immune response by an immunoreactive reagent in a subject comprising the steps of:
(a) administering to the subject a heat shock protein preparation; and
(b) administering to the subject a purified immunoreactive reagent that recognizes an antigen of a component against which an immune response is desired to be induced, the immunoreactive reagent being in an amount that is sub-optimal for said component in the absence of step (a),
such that an immune response is induced in the subject, and wherein the heat shock protein preparation does not display the antigenicity of the component.

8. (canceled)

9. A method of treating or preventing a cancer in a subject comprising the steps of:
(a) administering to the subject an immunoreactive reagent that recognizes an antigen associated with a cancer cell; and
(b) administering to the subject an amount of a heat shock protein preparation effective to induce or increase an immune response in the subject, wherein the heat shock protein preparation does not display the antigenicity of said cancer cell.

10. (canceled)

11. A method of immunotherapy comprising the steps of:
(a) administering to a subject an immunoreactive reagent that recognizes an antigen of a component against which an immune response is desired to be produced; and
(b) administering to the subject a heat shock protein preparation, wherein the heat shock protein preparation does not display the antigenicity of said component;
such that an immune response is produced in the subject.

12. A method of inducing an immune response by an immunoreactive reagent in a subject comprising the steps of:
(a) administering to the subject a heat shock protein preparation; and
(b) administering to the subject an immunoreactive reagent that recognizes an antigen of a component against which an immune response is desired to be induced, the immunoreactive reagent being in an amount that is sub-immunogenic for said component in the absence of step (a),
such that an immune response is induced in the subject, and wherein the heat shock protein preparation displays the antigenicity of said component.

13. (canceled)

14. A method of treating or preventing a cancer in a subject comprising the steps of:
(a) administering to the subject an immunoreactive reagent that recognizes an antigen associated with a cancer cell; and
(b) administering to the subject an amount of a heat shock protein preparation effective to induce or increase an immune response in the subject, wherein the heat shock protein preparation displays the antigenicity of said cancer cell.

15. (canceled)

16. The method of claims 9, 11, or 12, wherein the immune response produced in the subject as a result of the administration of said immunoreactive reagent is increased relative to said immune response in the absence of step (b).

17-58. (canceled)

59. A composition comprising a purified HSP preparation and one or more purified immunoreactive reagents.

60-61. (canceled)

62. A kit comprising:
(a) a first container containing a purified heat shock protein preparation in an amount effective to increase an immune response elicited by an immunoreactive reagent against a component of the immunoreactive reagent against which an immune response is desired; and
(b) a second container containing the immunoreactive reagent in purified form and in an amount that, when administered before, concurrently with, or after the administration of the heat shock protein preparation of (a), is effective to induce an immune response against the component.

63. The method of claim 1, 2, or 3 wherein said immunoreactive reagent is a prophylactic or therapeutic antibody.

64. The method of claim 1, 2, or 3 wherein said immunoreactive reagent is a monoclonal antibody.

65. The method of claim 1, 2, or 3 wherein said immunoreactive reagent is a polyclonal antibody.

66. The method of claim 1, 2, or 3 wherein said immune response is enhanced effector cell function.

67. The method of claim 1, 2, or 3 wherein said immune response is enhanced cytokine release.

68. The method of claim 1, 2, or 3 wherein said immune response is enhanced antibody-dependent cellular cytotoxicity or antibody-mediated opsonization or phagocytosis directed against a cell, pathogen, or protein possessing the epitope recognized by the antibody

69. The method of claim 1, 2, or 3 wherein said heat shock protein preparation comprises a heat shock protein selected from the group consisting of hsp60, hsp70, hsp90, gp96, calreticulin, or a combination thereof.

70. The method of claim 1, 2, or 3 wherein said heat shock protein preparation comprises a heat shock protein-peptide complex selected from the group consisting of hsp60-peptide complexes, hsp70-peptide complexes, hsp90-peptide complexes, gp96-peptide complexes, calreticulin-peptide complexes, or a combination thereof.

71. The method of claim 1, 2, or 3 wherein the heat shock protein preparation comprises heat shock protein-peptide complexes and purified heat shock proteins.

72-73. (canceled)

74. The method of claim 1, 2, or 3 wherein the subject is human and the heat shock protein preparation comprises mammalian heat shock proteins.

75. The method of claim 1, 2, or 3 wherein the heat shock protein preparation is administered before the administration of the immunoreactive reagent.

76. The method of claim 1, 2, or 3 wherein the heat shock protein preparation is administered concurrently with the administration of the immunoreactive reagent.

77. The method of claim 1, 2, or 3 wherein the heat shock protein is preparation administered after the administration of the immunoreactive reagent.

78. (canceled)

79. The method of claim 3, 9, 14, wherein the cancer is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, melanoma, neuroblastoma, retinoblastoma leukemia, acute lymphocytic leukemia, acute myelocytic leukemia acute myeloblastic leukemia, acute promyelocytic leukemia, acute myelomonocytic leukemia, acute monocytic leukemia, acute erythroleukemia, chronic leukemia chronic myelocytic leukemia, chronic granulocytic leukemia, chronic lymphocytic leukemia, polycythemia vera, lymphoma Hodgkin's disease lymphoma, non-Hodgkin's disease lymphoma, multiple myeloma, Waldenström's macroglobulinemia, and heavy chain disease.

80. (canceled)
